(19) United States
(12) Patent Application Publication
(10) Pub. No.: US 2010/0159597 A1
(43) Pub. Date: Jun. 24, 2010

(54) CELL CULTURE MEMBRANE, CELL CULTURE KIT, POROUS MATERIAL, PRODUCTION METHOD FOR CELL CULTURE MEMBRANE AND PRODUCTION METHOD FOR POROUS MATERIAL

(75) Inventors: Masatoshi Kuroda, Tokyo (JP); Youichi Takahashi, Shinjuku-Ku (JP); Naoya Ogata, Suginami-Ku (JP); Amane Nakamura, Suginami-Ku (JP); Yukari Yamazaki (formely Suzuki), Suginami-Ku (JP); Kanji Yamaoka, Suginami-Ku (JP)

Correspondence Address: BURR & BROWN, PO BOX 7068, SYRACUSE, NY, 13261-7068, US

(73) Assignee: DAI NIPPON PRINTING CO., LTD., SHINJUKU-KU TOKYO (JP)

(21) Appl. No.: 12/663,928

(22) PCT Filed: Jun. 11, 2008
(86) PCT No.: PCT/JP2008/060691

§ 371 Date: Jan. 25, 2010

(30) Foreign Application Priority Data
Jun. 11, 2007 (JP) 2007-153888
Sep. 28, 2007 (JP) 2007-256682
Oct. 10, 2007 (JP) 2007-264427

(51) Int. Cl. C12N 5/071

(52) U.S. Cl. 435/401

(57) ABSTRACT

To produce a cell culture membrane having biocompatibility utilizing DNA of natural resources and a cell culture kit, the cell culture membrane having DNA ionically-cross-linked with calcium ions or magnesium ions is provided. To produce a porous material utilizing DNA, a production method for a cell culture membrane and a production method for a porous material, fine pores of 1 nm to 100 μm in diameter are prepared in the porous material.

### TECHNICAL FIELD

The present invention relates to a cell culture technique utilizing a deoxyribonucleic acid (DNA), and more particularly relates to a cell culture membrane, a cell culture kit, a porous material, a production method for a cell culture membrane and a production method for a porous material.

### BACKGROUND ART

DNA is contained in salmon milt by about 10%. Most of salmon milt are abandoned and have given rise to an environmental problem, since salmon milt is not used as food sources. Hence, DNA of a natural resources is desirable to be utilized as a raw substance for industrial materials. Deoxyribonucleic acid (DNA) contained in a living body has an extremely high molecular weight. Hence, DNA can be prepared as a film by solution casting method and the like. However, a DNA film made by only casting is water-soluble, and has limited uses. Hence, a method for producing an water-insoluble DNA film has been proposed by mixing a sodium salt of DNA with an alkyl type quaternary ammonium cationic lipid (for example, refer to Patent Literature 1). DNA films produced by using an alkyl type quaternary ammonium cationic lipid have been utilized as a polarizing film.

Furthermore, DNA of a natural resources is desirable to be utilized for other purposes besides a polarizing film. Hence, DNA films were examined to utilize a cell culture membrane which is a basement membrane for a cell culture. However, DNA films produced by using an alkyl type quaternary ammonium cationic lipid have antibacterial and antifungal properties, and does not have biocompatibility. Hence, a conventional DNA film has been impossible to utilize as a cell culture membrane. Moreover, a porous material using DNA as a raw substance was difficult to produce. For example, when a porous material composed of DNA is produced by mixing DNA with a foamable material, and heating or doing a chemical treatment, there have been problems such as a denaturation of a double-helix structure or a degradation of DNA under a heating process at 100° C. or more or a chemical treatment process in an acidity or an alkaline environment.

### [Patent Literature 1] Japanese Patent Application Laid-Open Publication No. 8-239398

### DISCLOSURE OF THE INVENTION

### Problems to be Solved by the Invention

The purpose of the present invention is to provide a cell culture membrane having biocompatibility, a cell culture kit, a porous material making use of DNA, a production method for a cell culture membrane, and a production method for a porous material, utilizing DNA of a natural resource.

### Means for Solving the Problems

A first aspect of the present invention inheres in a cell culture membrane encompassing DNA ionically-cross-linked with calcium ions or magnesium ions. A cell culture membrane according to a first aspect has biocompatibility, and has insolubility to a cell culture medium for a certain period.

A second aspect of the present invention inheres in a cell culture membrane encompassing a salt of deoxyribonucleic acid and cations cross-linked by ultraviolet irradiation. A cell culture membrane according to a second aspect also has biocompatibility, and has insolubility to a cell culture medium for a certain period.

A third aspect of the present invention inheres in a cell culture kit encompassing (a) a cell culture membrane having DNA ionically-cross-linked with calcium ions or magnesium ions, and (b) cells adhered to the cell culture membrane.

A fourth aspect of the present invention inheres in a cell culture kit encompassing (a) a cell culture membrane having a salt of deoxyribonucleic acid and cations cross-linked by ultraviolet irradiation, and (b) cells adhered to the cell culture membrane.

A fifth aspect of the present invention inheres in a production method for a cell culture membrane encompassing (a) forming an water-soluble film containing a salt of DNA and monovalent cations, and (b) forming DNA ionically-cross-linked with calcium ions or magnesium ions by substituting monovalent cations with calcium ions or magnesium ions. According to a production method for a cell culture membrane according to a fifth aspect, a cell culture membrane having biocompatibility, and having insolubility to a cell culture medium for a certain period can be produced.

A sixth aspect of the present invention inheres in a production method for a cell culture membrane encompassing (a) forming an water-soluble film containing a salt of DNA and monovalent cations, and (b) irradiating the water-soluble film with ultraviolet. By adopting a production method for a cell culture membrane according to a sixth aspect, a cell culture membrane having biocompatibility, and having insolubility to a cell culture medium for a certain period can be produced.

A seventh aspect of the present invention inheres in a cell culture membrane encompassing deoxyribonucleic acid ionically-cross-linked with quaternary ammonium ions. A cell culture membrane according to a seventh aspect has also biocompatibility and insoluble to a cell culture medium for a certain period.

An eighth aspect of the present invention inheres in a cell culture kit encompassing (a) a cell culture membrane containing deoxyribonucleic acid ionically-cross-linked with quaternary ammonium ions, and (b) cells adhered to the cell culture membrane.

A ninth aspect of the present invention inheres in a production method for a cell culture membrane encompassing (a) forming an water-soluble film containing a salt of deoxyribonucleic acid and sodium ions, and (b) ionically-cross-linking the deoxyribonucleic acid with the quaternary ammonium ions by substituting sodium ions with quaternary ammonium ions. According to a production method for a cell culture membrane according to a ninth aspect, a cell culture membrane having biocompatibility and insoluble to a cell culture medium for a certain period can be produced.

A tenth aspect of the present invention inheres in a porous material composed of deoxyribonucleic acid, and having fine pores of 1 nm to 100 μm in diameter.

An eleventh aspect of the present invention inheres in a cell culture membrane encompassing (a) a porous material composed of cross-linked deoxyribonucleic acid, and having fine pores of 1 nm to 100 μm in diameter, and (b) collagen arranged in the fine pores of the porous material.

A twelfth aspect of the present invention inheres in a production method for a porous material encompassing (a) producing a mixture by mixing deoxyribonucleic acid and an alcoholic solubility polymer, and (b) forming a porous material composed of the deoxyribonucleic acid by dissolving the alcoholic soluble polymer contained in the mixture with alcohol.

A thirteenth aspect of the present invention inheres in a production method for a cell culture membrane encompassing (a) producing a mixture by mixing deoxyribonucleic acid and an alcoholic soluble polymer, (b) forming a porous material composed of the deoxyribonucleic acid by dissolving the alcoholic soluble polymer contained in the mixture with alcohol; (c) cross-linking the deoxyribonucleic acid contained in the porous material; and (d) impregnating collagen into the porous material.

### EFFECT OF THE INVENTION

According to the present invention, a cell culture membrane having biocompatibility, a cell culture kit, a porous material making use of DNA, a production method for a cell culture membrane and a production method for a porous material, utilizing DNA of a natural resource, can be provided.

### BRIEF DESCRIPTION OF THE DRAWINGS

FIG. 1 is a flow chart of a production method for a cell culture membrane according to a first embodiment of the present invention;

FIG. 2 is a view showing a frame format of a tube according to a first example of a first embodiment of the present invention;

FIG. 3 is a first view showing a frame format of a bottle according to a first example of a first embodiment of the present invention;

FIG. 4 is a second view showing a frame format of a bottle according to a first example of a first embodiment of the present invention;

FIG. 5 is a first view showing a frame format of a petri dish according to a first example of a first embodiment of the present invention;

FIG. 6 is a second view showing a frame format of a petri dish according to a first example of a first embodiment of the present invention;

FIG. 7 is a view showing a frame format of a petri dish and a cell culture membrane according to a first example of a first embodiment of the present invention;

FIG. 8 is a photograph of mouse chondrocytes (ATDC5) on a cell culture membrane composed of a calcium salt of DNA according to a first example of a first embodiment of the present invention;

FIG. 9 is a photograph of human hepatoma cells (Huh7) on a cell culture membrane composed of a magnesium salt of DNA according to a second example of a first embodiment of the present invention;

FIG. 10 is a first photograph of human hepatoma cells (Huh7) on a cell culture membrane composed of a calcium salt of DNA according to a second example of a first embodiment of the present invention;

FIG. 11 is a second photograph of human hepatoma cells (Huh7) on a cell culture membrane composed of a calcium salt of DNA according to a second example of a first embodiment of the present invention;

FIG. 12 is a view showing a frame format of a petri dish and a cell culture membrane according to a third example of a first embodiment of the present invention;

FIG. 13 is a photograph of mouse bone-marrow-derived undifferentiated mesenchymal cells (C3H10T1/2) on a cell culture membrane composed of a calcium salt of DNA according to a third example of a first embodiment of the present invention;

FIG. 14 is a first photograph of mouse chondrocytes (ATDC5) on a cell culture membrane composed of a photoproduct of DNA according to a first example of a second embodiment of the present invention;

FIG. 15 is a second photograph of mouse chondrocytes (ATDC5) on a cell culture membrane composed of a photoproduct of DNA according to a first example of a second embodiment of the present invention;

FIG. 16 is a third photograph of mouse chondrocytes (ATDC5) on a cell culture membrane composed of a photoproduct of DNA according to a first example of a second embodiment of the present invention;

FIG. 17 is a first photograph of mouse bone-mallow-derived undifferentiated mesenchymal cells (C3H10T1/2) on a cell culture membrane composed of a photoproduct according to a second example of a second embodiment of the present invention;

FIG. 18 is a second photograph of mouse bone-mallow-derived undifferentiated mesenchymal cells (C3H10T1/2) on a cell culture membrane composed of a photoproduct according to a second example of a second embodiment of the present invention;

FIG. 19 is a third photograph of mouse bone-mallow-derived undifferentiated mesenchymal cells (C3H10T1/2) on a cell culture membrane composed of a photoproduct according to a second example of a second embodiment of the present invention;

FIG. 20 is a photograph of mouse chondrocytes (ATDC5) on a cell culture membrane composed of a calcium salt of a mixture of DNA and salmon-derived type I collagen according to a first example of a third embodiment of the present invention;

FIG. 21 is a first photograph of mouse chondrocytes (ATDC5) on a cell culture membrane composed of a mixture of a calcium salt of DNA and salmon-derived type I collagen according to a first example of a third embodiment of the present invention;

FIG. 22 is the second photograph of mouse chondrocytes (ATDC5) on a cell culture membrane composed of a mixture of a calcium salt of DNA and salmon-derived type I collagen according to a first example of a third embodiment of the present invention;

FIG. 23 is a first photograph of mouse chondrocytes (ATDC5) on a cell culture membrane composed of a mixture of a calcium salt of DNA and salmon-derived type I collagen according to a first example of a third embodiment of the present invention;

FIG. 24 is a second photograph of mouse chondrocytes (ATDC5) on a cell culture membrane composed of a mixture of a calcium salt of DNA and salmon-derived type I collagen according to a second example of a third embodiment of the present invention;

FIG. 25 is a third photograph of mouse chondrocytes (ATDC5) on a cell culture membrane composed of a mixture of a calcium salt of DNA and salmon-derived type I collagen according to a second example of a third embodiment of the present invention;

FIG. 26 is a first photograph of mouse chondrocytes (ATDC5) on a cell culture membrane composed of a calcium salt of a mixture of DNA and salmon-derived type I collagen according to a second example of a third embodiment of the present invention;

FIG. 27 is a second photograph of mouse chondrocytes (ATDC5) on a cell culture membrane composed of a calcium salt of a mixture of DNA and salmon-derived type I collagen according to a second example of a third embodiment of the present invention;

FIG. 28 is a third photograph of mouse chondrocytes (ATDC5) on a cell culture membrane composed of a calcium salt of a mixture of DNA and salmon-derived type I collagen according to a second example of a third embodiment of the present invention;

FIG. 29 is a first photograph of mouse bone-mallow-derived undifferentiated mesenchymal cells (C3H10T1/2) on a cell culture membrane composed of a calcium salt of a mixture of DNA and salmon-derived type I collagen according to a third example of a third embodiment of the present invention;

FIG. 30 is a second photograph of mouse bone-mallow-derived undifferentiated mesenchymal cells (C3H10T1/2) on a cell culture membrane composed of a calcium salt of a mixture of DNA and salmon-derived type I collagen according to a third example of a third embodiment of the present invention;

FIG. 31 is a first photograph of mouse bone-mallow-derived undifferentiated mesenchymal cells (C3H10T1/2) on a cell culture membrane composed of a mixture of a calcium salt of DNA and salmon-derived type I collagen according to a third example of a third embodiment of the present invention;

FIG. 32 is a second photograph of mouse bone-mallow-derived undifferentiated mesenchymal cells (C3H10T1/2) on a cell culture membrane composed of a mixture of a calcium salt of DNA and salmon-derived type I collagen according to a third example of a third embodiment of the present invention;

FIG. 33 is a first photograph of mouse bone-mallow-derived undifferentiated mesenchymal cells (C3H10T1/2) on a cell culture membrane composed of a mixture of a calcium salt of DNA and salmon-derived type I collagen according to a fourth example of a third embodiment of the present invention;

FIG. 34 is a second photograph of mouse bone-mallow-derived undifferentiated mesenchymal cells (C3H10T1/2) on a cell culture membrane composed of a mixture of a calcium salt of DNA and salmon-derived type I collagen according to a fourth example of a third embodiment of the present invention;

FIG. 35 is a third photograph of mouse bone-mallow-derived undifferentiated mesenchymal cells (C3H10T1/2) on a cell culture membrane composed of a mixture of a calcium salt of DNA and salmon-derived type I collagen according to a fourth example of a third embodiment of the present invention;

FIG. 36 is a first photograph of mouse bone-mallow-derived undifferentiated mesenchymal cells (C3H10T1/2) on a cell culture membrane composed of a calcium salt of a mixture of DNA and salmon-derived type I collagen according to a fourth example of a third embodiment of the present invention;

FIG. 37 is a second photograph of mouse bone-mallow-derived undifferentiated mesenchymal cells (C3H10T1/2) on a cell culture membrane composed of a calcium salt of a mixture of DNA and salmon-derived type I collagen according to a fourth example of a third embodiment of the present invention;

FIG. 38 is a third photograph of mouse bone-mallow-derived undifferentiated mesenchymal cells (C3H10T1/2) on a cell culture membrane composed of a calcium salt of a mixture of DNA and salmon-derived type I collagen according to a fourth example of a third embodiment of the present invention;

FIG. 39 is a flow chart of a production method for a porous material according to a fifth embodiment of the present invention;

FIG. 40 is a first photograph showing a film composed of a mixture material according to a fifth embodiment of the present invention;

FIG. 41 is a second photograph showing a film composed of a mixture material according to a fifth embodiment of the present invention;

FIG. 42 is a third photograph showing a film composed of a mixture material according to a fifth embodiment of the present invention;

FIG. 43 is a first photograph showing a porous material according to a fifth embodiment of the present invention;

FIG. 44 is a second photograph showing a porous material according to a fifth embodiment of the present invention;

FIG. 45 is a third photograph showing a porous material according to a fifth embodiment of the present invention;

FIG. 46 is a first photograph showing a surface of a porous material according to a first example of a fifth embodiment of the present invention;

FIG. 47 is a second photograph showing a surface of a porous material according to a first example of a fifth embodiment of the present invention;

FIG. 48 is a third photograph showing a surface of a porous material according to a first example of a fifth embodiment of the present invention;

FIG. 49 is a first photograph showing a back side of a porous material according to a first example of a fifth embodiment of the present invention;

FIG. 50 is a second photograph showing a back side of a porous material according to a first example of a fifth embodiment of the present invention;

FIG. 51 is a third photograph showing a back side of a porous material according to a first example of a fifth embodiment of the present invention;

FIG. 52 is a first photograph showing a surface of a porous material according to a second example of a fifth embodiment of the present invention;

FIG. 53 is a second photograph showing a surface of a porous material according to a second example of a fifth embodiment of the present invention;

FIG. 54 is a third photograph showing a surface of a porous material according to a second example of a fifth embodiment of the present invention;

FIG. 55 is a first photograph showing a back side of a porous material according to a second example of a fifth embodiment of the present invention;

FIG. 56 is a second photograph showing a back side of a porous material according to a second example of a fifth embodiment of the present invention;

FIG. 57 is a third photograph showing a back side of a porous material according to a second example of a fifth embodiment of the present invention;

FIG. 58 is a first photograph showing a surface of a film composed of a mixture of DNA and PNIPAAm according to a third example of a fifth embodiment of the present invention;

FIG. 59 is a second photograph showing a surface of a film composed of a mixture of DNA and PNIPAAm according to a third example of a fifth embodiment of the present invention;

FIG. 60 is a third photograph showing a surface of a film composed of a mixture of DNA and PNIPAAm according to a third example of a fifth embodiment of the present invention;

FIG. 61 is a first photograph showing a back side of a film composed of a mixture of DNA and PNIPAAm according to a third example of a fifth embodiment of the present invention;

FIG. 62 is a second photograph showing a back side of a film composed of a mixture of DNA and PNIPAAm according to a third example of a fifth embodiment of the present invention;

FIG. 63 is a third photograph showing a back side of a film composed of a mixture of DNA and PNIPAAm according to a third example of a fifth embodiment of the present invention;

FIG. 64 is a first photograph showing a surface of a porous material according to a third example of a fifth embodiment of the present invention;

FIG. 65 is a second photograph showing a surface of a porous material according to a third example of a fifth embodiment of the present invention;

FIG. 66 is a third photograph showing a surface of a porous material according to a third example of a fifth embodiment of the present invention;

FIG. 67 is a first photograph showing a back side of a porous material according to a third example of a fifth embodiment of the present invention;

FIG. 68 is a second photograph showing a back side of a porous material according to a third example of a fifth embodiment of the present invention;

FIG. 69 is a third photograph showing a back side of a porous material according to a third example of a fifth embodiment of the present invention;

FIG. 70 is a first photograph showing a surface of a film composed of a mixture of DNA and PNIPAAm according to a fourth example of a fifth embodiment of the present invention;

FIG. 71 is a second photograph showing a surface of a film composed of a mixture of DNA and PNIPAAm according to a fourth example of a fifth embodiment of the present invention;

FIG. 72 is a third photograph showing a surface of a film composed of a mixture of DNA and PNIPAAm according to a fourth example of a fifth embodiment of the present invention;

FIG. 73 is a first photograph showing a back side of a film composed of a mixture of DNA and PNIPAAm according to a fourth example of a fifth embodiment of the present invention;

FIG. 74 is a second photograph showing a back side of a film composed of a mixture of DNA and PNIPAAm according to a fourth example of a fifth embodiment of the present invention;

FIG. 75 is a third photograph showing a back side of a film composed of a mixture of DNA and PNIPAAm according to a fourth example of a fifth embodiment of the present invention;

FIG. 76 is a first photograph showing a surface of a porous material according to a fourth example of a fifth embodiment of the present invention;

FIG. 77 is a second photograph showing a surface of a porous material according to a fourth example of a fifth embodiment of the present invention;

FIG. 78 is a third photograph showing a surface of a porous material according to a fourth example of a fifth embodiment of the present invention;

FIG. 79 is a first photograph showing a back side of a porous material according to a fourth example of a fifth embodiment of the present invention;

FIG. 80 is a second photograph showing a back side of a porous material according to a fourth example of a fifth embodiment of the present invention;

FIG. 81 is a third photograph showing a back side of a porous material according to a fourth example of a fifth embodiment of the present invention;

FIG. 82 is a first photograph showing a surface of a film composed of a mixture of DNA and PNIPAAm according to a fifth example of a fifth embodiment of the present invention;

FIG. 83 is a second photograph showing a surface of a film composed of a mixture of DNA and PNIPAAm according to a fifth example of a fifth embodiment of the present invention;

FIG. 84 is a third photograph showing a surface of a film composed of a mixture of DNA and PNIPAAm according to a fifth example of a fifth embodiment of the present invention;

FIG. 85 is a first photograph showing a back side of a film composed of a mixture of DNA and PNIPAAm according to a fifth example of a fifth embodiment of the present invention;

FIG. 86 is a second photograph showing aback side of a film composed of a mixture of DNA and PNIPAAm according to a fifth example of a fifth embodiment of the present invention;

FIG. 87 is a third photograph showing a back side of a film composed of a mixture of DNA and PNIPAAm according to a fifth example of a fifth embodiment of the present invention;

FIG. 88 is a first photograph showing a surface of a porous material according to a fifth example of a fifth embodiment of the present invention;

FIG. 89 is a second photograph showing a surface of a porous material according to a fifth example of a fifth embodiment of the present invention;

FIG. 90 is a third photograph showing a surface of a porous material according to a fifth example of a fifth embodiment of the present invention;

FIG. 91 is a first photograph showing aback side of a porous material according to a fifth example of a fifth embodiment of the present invention;

FIG. 92 is a second photograph showing a back side of a porous material according to a fifth example of a fifth embodiment of the present invention;

FIG. 93 is a third photograph showing a back side of a porous material according to a fifth example of a fifth embodiment of the present invention;

FIG. 94 is a first photograph showing a surface of a film composed of a mixture of DNA and PNIPAAm according to a sixth example of a fifth embodiment of the present invention;

FIG. 95 is a second photograph showing a surface of a film composed of a mixture of DNA and PNIPAAm according to a sixth example of a fifth embodiment of the present invention;

FIG. 96 is a third photograph showing a surface of a film composed of a mixture of DNA and PNIPAAm according to a sixth example of a fifth embodiment of the present invention;

FIG. 97 is a first photograph showing a back side of a film composed of a mixture of DNA and PNIPAAm according to a sixth example of a fifth embodiment of the present invention;

FIG. 98 is a second photograph showing a back side of a film composed of a mixture of DNA and PNIPAAm according to a sixth example of a fifth embodiment of the present invention;

FIG. 99 is a first photograph showing a surface of a porous material according to a sixth example of a fifth embodiment of the present invention;

FIG. 100 is a second photograph showing a surface of a porous material according to a sixth example of a fifth embodiment of the present invention;

FIG. 101 is a third photograph showing a surface of a porous material according to a sixth example of a fifth embodiment of the present invention;

FIG. 102 is a first photograph showing a back side of a porous material according to a sixth example of a fifth embodiment of the present invention;

FIG. 103 is a second photograph showing a back side of a porous material according to a sixth example of a fifth embodiment of the present invention;

FIG. 104 is a third photograph showing a back side of a porous material according to a sixth example of a fifth embodiment of the present invention;

FIG. 105 is a first photograph showing a surface of a film composed of a mixture of DNA and PVP according to a seventh example of a fifth embodiment of the present invention;

FIG. 106 is a second photograph showing a surface of a film composed of a mixture of DNA and PVP according to a seventh example of a fifth embodiment of the present invention;

FIG. 107 is a third photograph showing a surface of a film composed of a mixture of DNA and PVP according to a seventh example of a fifth embodiment of the present invention;

FIG. 108 is a first photograph showing a surface of a porous material according to a seventh example of a fifth embodiment of the present invention;

FIG. 109 is a second photograph showing a surface of a porous material according to a seventh example of a fifth embodiment of the present invention;

FIG. 110 is a third photograph showing a surface of a porous material according to a seventh example of a fifth embodiment of the present invention;

FIG. 111 is a first photograph showing a surface of a film composed of a mixture of DNA and PVP according to an eighth example of a fifth embodiment of the present invention;

FIG. 112 is a second photograph showing a surface of a film composed of a mixture of DNA and PVP according to an eighth example of a fifth embodiment of the present invention;

FIG. 113 is a third photograph showing a surface of a film composed of a mixture of DNA and PVP according to an eighth example of a fifth embodiment of the present invention;

FIG. 114 is a first photograph showing a surface of a porous material according to an eighth example of a fifth embodiment of the present invention;

FIG. 115 is a second photograph showing a surface of a porous material according to an eighth example of a fifth embodiment of the present invention;

FIG. 116 is a third photograph showing a surface of a porous material according to an eighth example of a fifth embodiment of the present invention;

FIG. 117 is a first photograph showing a surface of a film composed of a mixture of DNA and PVP according to a ninth example of a fifth embodiment of the present invention;

FIG. 118 is a second photograph showing a surface of a film composed of a mixture of DNA and PVP according to a ninth example of a fifth embodiment of the present invention;

FIG. 119 is a third photograph showing a surface of a film composed of a mixture of DNA and PVP according to a ninth example of a fifth embodiment of the present invention;

FIG. 120 is a first photograph showing a surface of a porous material according to a ninth example of a fifth embodiment of the present invention;

FIG. 121 is a second photograph showing a surface of a porous material according to a ninth example of a fifth embodiment of the present invention;

FIG. 122 is a third photograph showing a surface of a porous material according to a ninth example of a fifth embodiment of the present invention;

FIG. 123 is a first photograph showing aback side of a porous material according to a ninth example of a fifth embodiment of the present invention;

FIG. 124 is a second photograph showing a back side of a porous material according to a ninth example of a fifth embodiment of the present invention;

FIG. 125 is a third photograph showing a back side of a porous material according to a ninth example of a fifth embodiment of the present invention;

FIG. 126 is a third view showing a frame format of a petri dish according to an example of a sixth embodiment of the present invention;

FIG. 127 is a photograph of mouse chondrocytes (ATDC5) on a porous material composed of a calcium salt of DNA according to an example of a sixth embodiment of the present invention;

FIG. 128 is a photograph of mouse chondrocytes (ATDC5) on a flat film composed of a calcium salt of DNA according to a comparative example of a sixth embodiment of the present invention;

FIG. 129 is a flow chart of a test method on usefulness of a filter according to a first example of an eighth embodiment of the present invention;

FIG. 130 is a graph showing a test result on usefulness of a filter according to a first example of an eighth embodiment of the present invention;

FIG. 131 is a flow chart of a test method on usefulness of a filter according to a second example of an eighth embodiment of the present invention;

FIG. 132 is a graph showing a test result on usefulness of a filter according to a second example of an eighth embodiment of the present invention.

### BEST MODE FOR CARRYING OUT THE INVENTION

A description will be made below of an embodiment of the present invention. In the following description made with reference to the drawings, the same or similar portions are denoted by the same or similar reference numerals. Note that the drawings are schematic. Hence, specific dimensions and the like should be determined with reference to the following description. Moreover, it is a matter of course that portions different in dimensional relation ship and ratio from one another are also included in the drawings.

### First Embodiment

A cell culture membrane according to a first embodiment is composed of deoxyribonucleic acid (DNA) ionically-cross-linked with calcium ions (Ca²⁺) or magnesium ions (Mg²⁺). Cell culture membranes composed of DNA ionically-cross-linked with calcium ions and of DNA ionically-cross-linked with magnesium ions are transparent, respectively, and are suitable for cell observation with an optical microscope. A cell culture membrane according to a first embodiment has biocompatibility and high affinity for a cell. Moreover, a cell culture membrane according to a first embodiment is not soluble in a cell culture medium for two days or more, and possible to preserve its form.

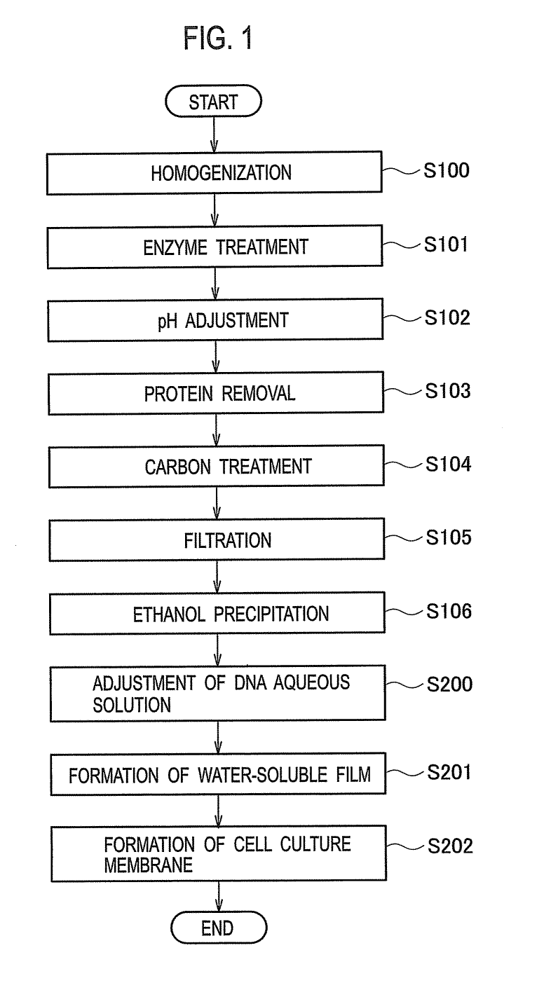

Next, using a flow chart shown in FIG. 1, a production method for a cell culture membrane according to a first embodiment will be explained.

(a) At a step S100, salmon milt, mid-gut gland of scallop or the like is homogenized with a homogenizer. Next, salmon milt or mid-gut gland of scallop homogenized is filtrated, and a first filtrate is obtained. At a step S101, a proteolytic enzyme e.g. protease and the like is added to a first filtrate, proteins contained in a first filtrate are degraded. Then, at a step S102, pH of a first filtrate is adjusted, and at a step S103, proteins contained in a first filtrate are removed. At a step S104, a first filtrate is carbon-treated. At a step S105, a first filtrate is filtrated, and a second filtrate is obtained. At a step S106, ethanol is added to a second filtrate, a purified sodium salt of DNA that is a salt of DNA and monovalent cations is extracted. Then, a sodium salt of DNA is dried. Note that, DNA may be either a single strand or a double strand. Molecular weight of DNA is, for example, 100 kDa to 10,000 kDa. In the following example, a highly-pure DNA of an average molecular weight of 6,600 kDa and a purity of 90% or more is used.

(b) At a step S200, a sodium salt of DNA is dissolved in ultrapure water, and a DNA aqueous solution is adjusted up to 7.5 g/l concentration. At a step 201, a DNA aqueous solution is dropped on a plane e.g. a basal plane of petri dish of 100 mm in diameter and the like, and dried, then, an water-soluble film composed of a sodium salt of DNA is formed on a basal plane of petri dish. At a step 202, a calcium chloride (CaCl₂) aqueous solution of 1 mol/l is dropped on the petri dish, and an water-soluble film is immersed into a CaCl₂ aqueous solution for 3 hours or more. By immersing an water-soluble film into a CaCl₂ aqueous solution, sodium ions are substituted with calcium ions, and DNA is ionically-cross-linked with calcium ions. A gelled DNA ionically-cross-linked with calcium ions is collected as a cell culture membrane, a production method for a cell culture membrane according to a first embodiment is ended.

Note that, when a commercial DNA is used, a step S100 to a step S106 may be omitted. Moreover, at a step 202, an water-soluble film is immersed into a magnesium chloride (MgCl₂) aqueous solution of 1 mol/l instead of a CaCl₂ aqueous solution, then, a cell culture membrane composed of DNA ionically-cross-linked with magnesium ions can be produced. After a cell culture membrane is collected, the cell culture membrane may be rinsed with ultrapure water, and dried.

### First Example of First Embodiment

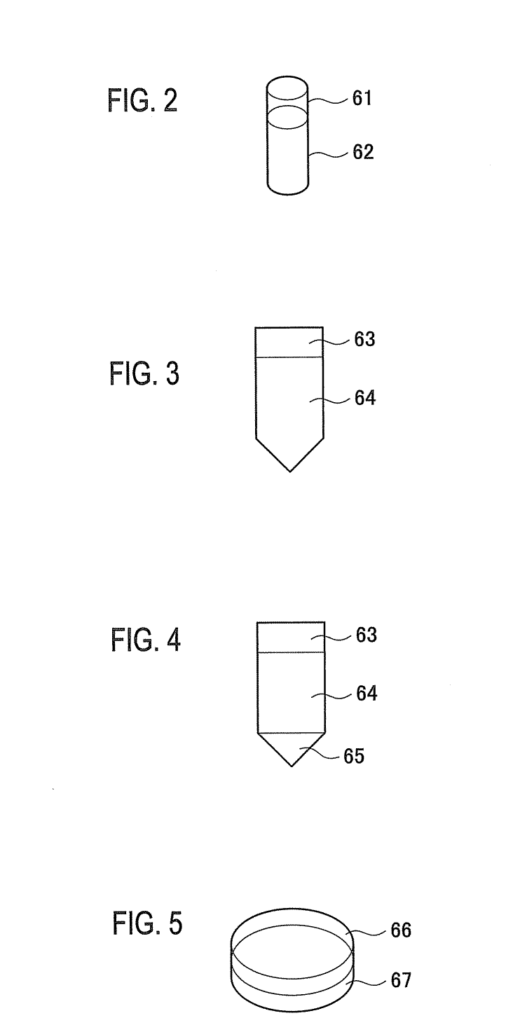

First, a fetal bovine serum (FBS) was added into a dulbecco modified eagle medium (DMEM) of 500 ml so as to be 10% in volume concentration, in addition, penicillin streptomycin was added so as to be 1% in volume concentration, thus a cell culture medium was prepared. Next, a cell cryopreserved solution 62 containing mouse chondrocytes (ATDC5) frozen at −80° C. in a cryopreservation tube 61 shown in FIG. 2 was defrosted. A defrosted cell cryopreseved solution 62 was dropped into a bottle 63 shown in FIG. 3, further, a cell culture medium was dropped into the bottle 63. After that, mouse chondrocytes were suspended with a cell culture medium, and a first suspension 64 was obtained.

The bottle 63 was rotated at rotation speed of 2000 rpm for 5 minutes, as shown in FIG. 4, mouse chondrocytes 65 contained in the first suspension 64 were precipitated. Next, a precipitated mouse chondrocytes 65 were suspended with a cell culture medium, a second suspension was obtained. After that, as shown in FIG. 5, a second suspension 67 was dropped on a petri dish 66 of 100 mm in diameter, mouse chondrocytes were cultured at 37° C. in carbon dioxide (CO₂) concentration of 5%. After mouse chondrocytes became confluent on a basal plane of the petri dish 66, a cell culture medium in the petri dish 66 was removed with suction. Next, mouse chondrocytes were washed with a phosphate buffered saline (PBS), and after washing, a phosphate buffered saline was removed with suction. After that, trypsin-EDTA of 5 ml was dropped on the petri dish 66, and incubated at 37° C. for 5 minutes. Mouse chondrocytes exfoliated by trypsin-EDTA from the petri dish 66 were centrifuged, and suspended with a cell culture medium.

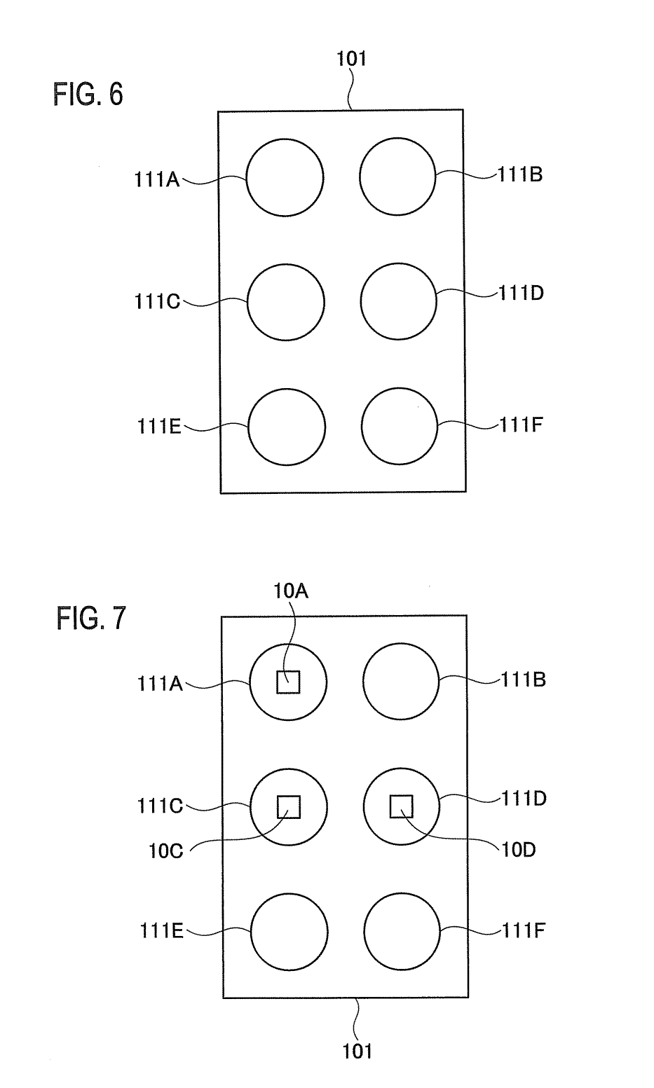

Next, as shown in FIG. 6, a petri dish 101 having six wells 111A, 111B, 111C, 111D, 111E, and 111F was prepared. Moreover, as shown in FIG. 7, a cell culture membrane 10A composed of DNA ionically-cross-linked with magnesium ions was set on a basal plane of well 111A, and cell culture membranes 100 and 10D composed of DNA ionically-cross-linked with calcium ions were set on basal planes of wells 111C and 111D, respectively. Note that, before setting on basal planes of wells 111A, 111B, and 111C, the cell culture membranes 10A, 100 and 10D were sterilized with ethanol of 70% volume concentration for 30 minutes. After that, the wells 111A to 111F were filled with a cell culture medium, and dispersed with mouse chondrocytes.

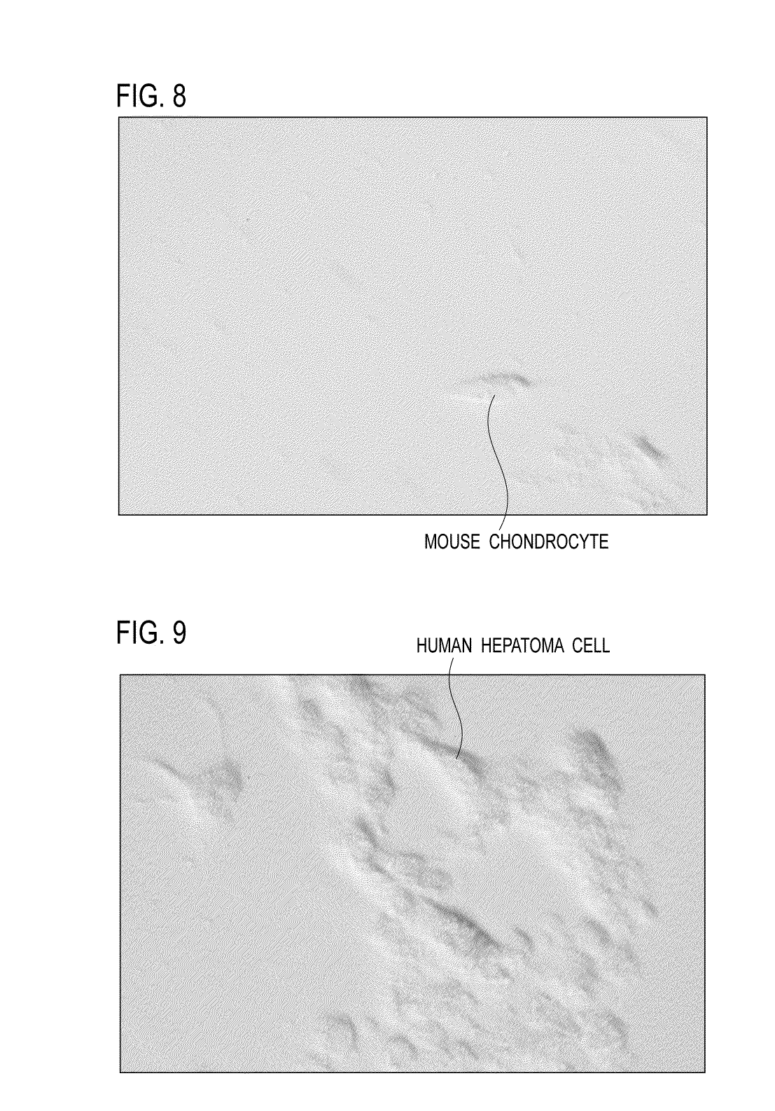

Mouse chondrocytes were adhered to the cell culture membrane 10A composed of DNA ionically-cross-linked with magnesium ions. The cell culture membrane 10A was dissolved in a cell culture medium, and died out after three days since a cell culture medium was filled in the well 111A. However, mouse chondrocytes were adhered to the cell culture membrane 10A until the cell culture membrane 10A was dissolved and died out. As shown in FIG. 8, mouse chondrocytes were also adhered and increased on the cell culture membranes 10C and 10D composed of DNA ionically-cross-linked with calcium ions. Note that, a photograph in FIG. 8 was taken after one day since mouse chondrocytes were dispersed. The cell culture membranes 100 and 10D were not dissolved into a cell culture medium. From results above, it was shown that a cell culture membrane composed of DNA ionically-cross-linked with magnesium ions or calcium ions has biocompatibility, and can be transplanted into a living body. Moreover, it was shown that after transplanting into a living body, in case of being suitable to be dissolved in a short time, a cell culture membrane composed of DNA ionically-cross-linked with magnesium ions is useful, and in case of being suitable to be dissolved in a long time, a cell culture membrane composed of DNA ionically-cross-linked with calcium ions is useful.

### Second Example of First Embodiment

A similar way to a first example of a first embodiment, cell culture membranes 10A, 100, and 10D were set on basal planes of wells 111A, 111C, and 111D on the petri dish 101 shown in FIG. 7, respectively. After that, the same cell culture medium as that of a first example was filled in wells of 111A to 111F, respectively, and dispersed with human hepatoma cells (Huh7).

As shown in FIG. 9, human hapatoma cells were adhered to the cell culture membrane 10A composed of DNA ionically-cross-linked with magnesium ions. Note that, a photograph of FIG. 9 was taken after one day since human hepatoma cells were dispersed. The cell culture membrane 10A was dissolved and died out after three days since a cell culture medium was filled in the well 111A.

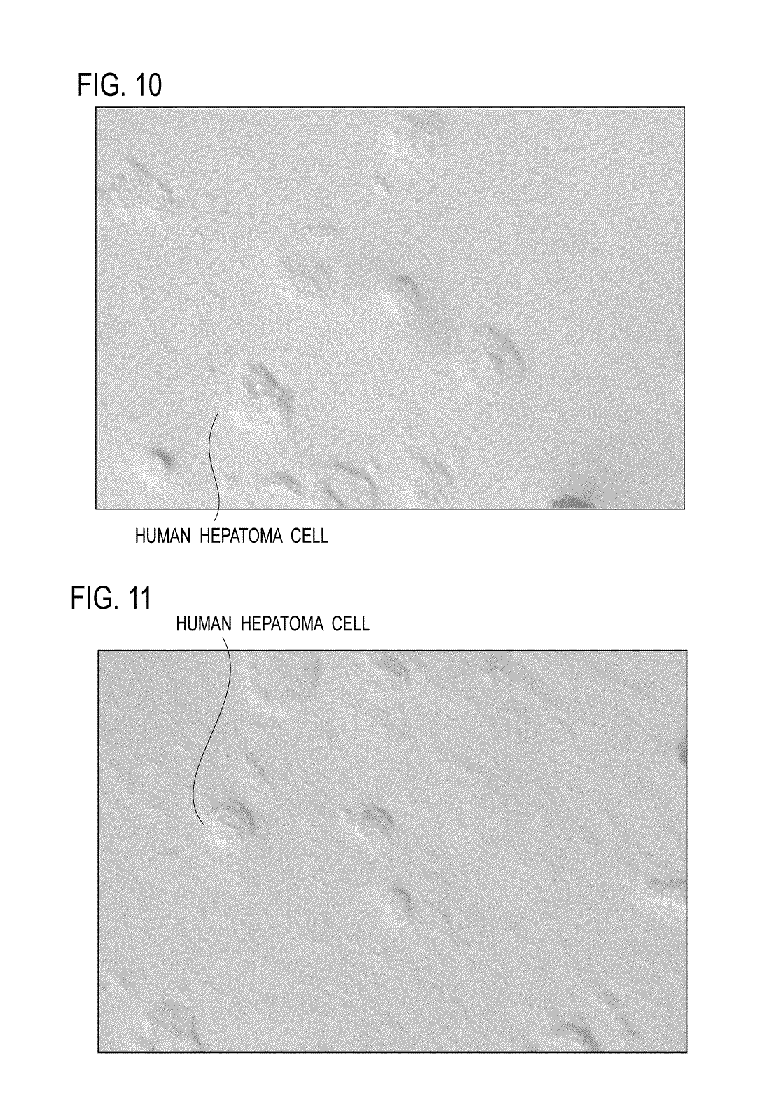

As shown in FIG. 10, human hepatoma cells were also adhered to the cell culture membranes 100 and 10D composed of DNA ionically-cross-linked with calcium ions. Note that, a photograph of FIG. 10 was taken after one day since human hepatoma cells were dispersed. And, as shown in FIG. 11, human hepatoma cells were still adhered on the cell culture membranes 10C and 10D after six days since human hepatoma cells had been dispersed. Although the cell culture membranes 10C and 10D had been gradually dissolved into a cell culture medium, they still existed in a cell culture medium after one week since a culture medium had been filled in the well 111A.

### Third Example of First Embodiment

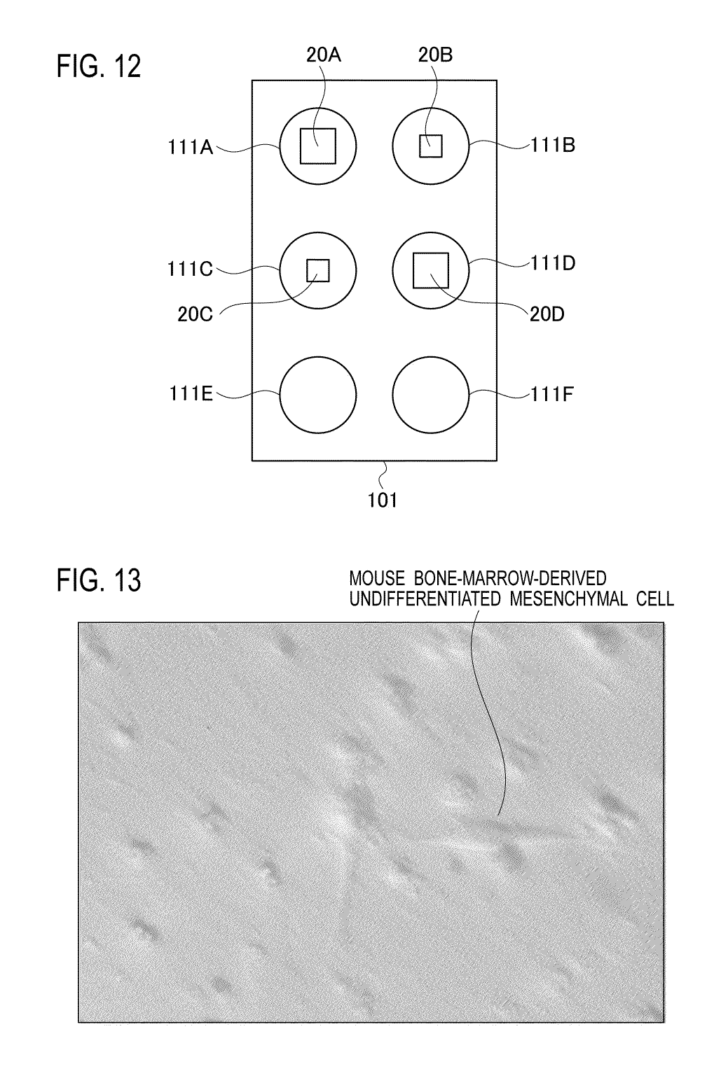

As shown in FIG. 12, a cell culture membrane 20A composed of DNA ionically-cross-linked with calcium ions was set on a basal plane of well 111A of petri dish 101. Moreover, on a basal plane of well 111B, a cell culture membrane 20B of smaller than 20A and composed of DNA ionically-cross-linked with calcium ions was set. Moreover, on a basal plane of well 111C, a cell culture membrane 20C of smaller than 20B and composed of DNA ionically-cross-linked with calcium ions was set. Moreover, on a basal plane of well 111D, a cell culture membrane 20D of the same size as 20B and composed of DNA ionically-cross-linked with calcium ions was set. After that, the same cell culture medium as that of a first example was filled in wells 111A to 111F, respectively, and dispersed with mouse bone-mallow-derived undifferentiated mesenchymal cells (C3H10T1/2).

Mouse bone-mallow-derived undifferentiated mesenchymal cells were adhered to the cell culture membranes 20A to 20D, respectively. Moreover, as shown in FIG. 13, mouse bone-mallow-derived undifferentiated mesenchymal cells were adhered on the cell culture membranes 20A to 20D, respectively, after five days since mouse bone-mallow-derived undifferentiated mesenchymal cells had been dispersed. A difference of area between each cell culture membrane of 20 A to 20D did not give any effect on an adhesiveness in a mouse bone-mallow-derived undifferentiated mesenchymal cell.

### Comparative Example of First Embodiment

A cell culture membrane composed of DNA ionically-cross-linked with copper ions (Cu²⁺) was set on a basal plane of well 111A of Petri dish 101, a cell culture membrane composed of DNA ionically-cross-linked with zinc ions (Zn²⁺) was set on a basal plane of well 111B, a cell culture membrane composed of DNA ionically-cross-linked with ferric ions (Fe³⁺) was set on a basal plane of well 111C, and a cell culture membrane composed of DNA ionically-cross-linked with ferrous ions (Fe²⁺) was set on a basal plane of well 111D. After that, wells 111A to 111F were filled with the same cell culture medium as that of a first example, respectively, and dispersed with mouse chondrocytes (ATDC5). After one day since mouse chondrocytes had been dispersed, the wells 111A to 111F were observed.

On a cell culture membrane composed of DNA ionically-cross-linked with copper ions, mouse chondrocytes were not adhered. Note that, a cell culture membrane composed of DNA ionically-cross-linked with copper ions was not dissolved in a cell culture medium. On a cell culture membrane composed of DNA ionically-cross-linked with zinc ions, mouse chondrocytes were not also adhered. Moreover, a cell culture membrane composed of DNA ionically-cross-linked with zinc ions were not dissolved in a cell culture medium, though a color was changed from colorless and transparent to white after one night since a cell culture membrane had been immersed in a cell culture medium.

Mouse chondrocytes were also not adhered to a cell culture membrane composed of DNA ionically-cross-linked with ferric ions. A cell culture membrane composed of DNA ionically-cross-linked with ferric ions begun to be dissolved just after immersing in a cell culture medium, and changed the cell culture medium to yellow. Mouse chondrocytes were also not adhered to a cell culture membrane composed of DNA ionically-cross-linked with ferrous ions. A cell culture membrane composed of DNA ionically-cross-linked with ferrous ions begun to dissolve after three days and changed a cell culture medium to brown.

### Second Embodiment

A cell culture membrane according to a second embodiment is composed of a DNA photoproduct. A DNA photoproduct is, say, a salt of deoxyribonucleic acid and monovalent cations cross-linked by ultraviolet (UV) irradiation. A DNA photoproduct is insoluble to water (H₂O). Next, a production method for a cell culture membrane according to a second embodiment will be explained. First, in the same way as a first embodiment, for example, an water-soluble film made of a sodium salt of DNA is formed on a basal plane of a petri dish. After that, UV of 1×10⁻² mJ/mm² or more is irradiated to an water-soluble film at room temperature. With UV irradiation, for example, thymines contained in an water-soluble film are cross-linked with each other, and thymine dimer is formed. Moreover, with UV irradiation, a part of enzyme of thymine (quinone structure) is cross-linked by reacting with other thymine or cytosine. Otherwise, a carbon-hydrogen bond (C—H) of ribose contained in DNA is cut with UV irradiation, a carbon radical reacts with other carbon-hydrogen bond. As a result, a cell culture membrane is formed from a salt of deoxyribonucleic acid photo-cross-linked with monovalent cations.

### First Example of Second Embodiment

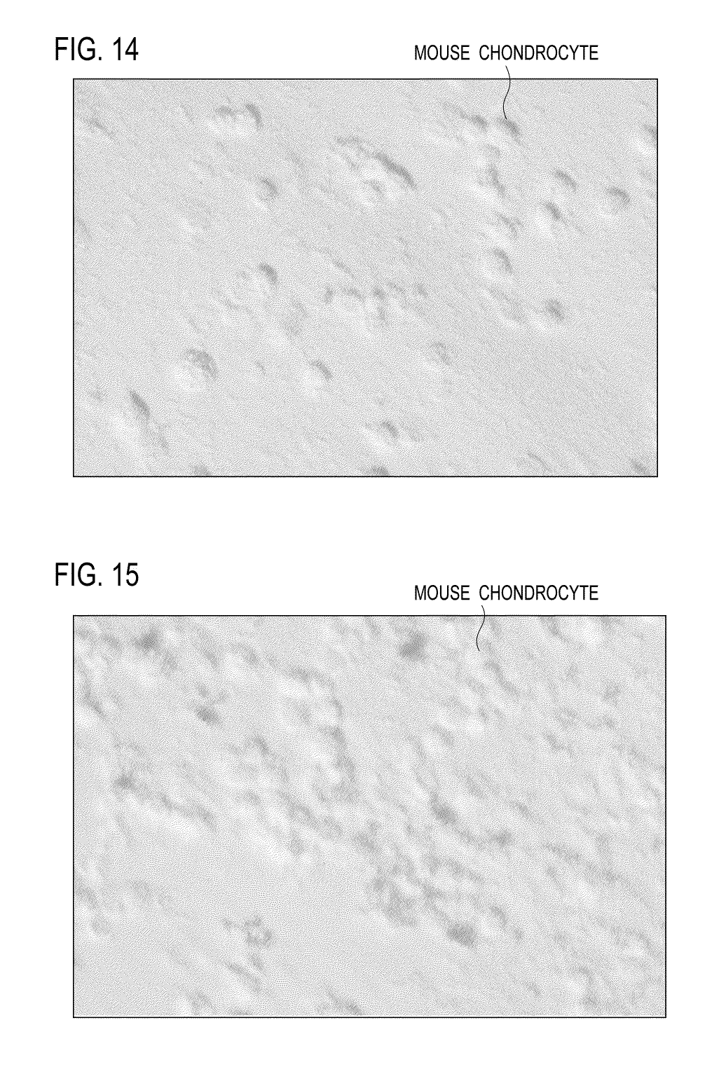

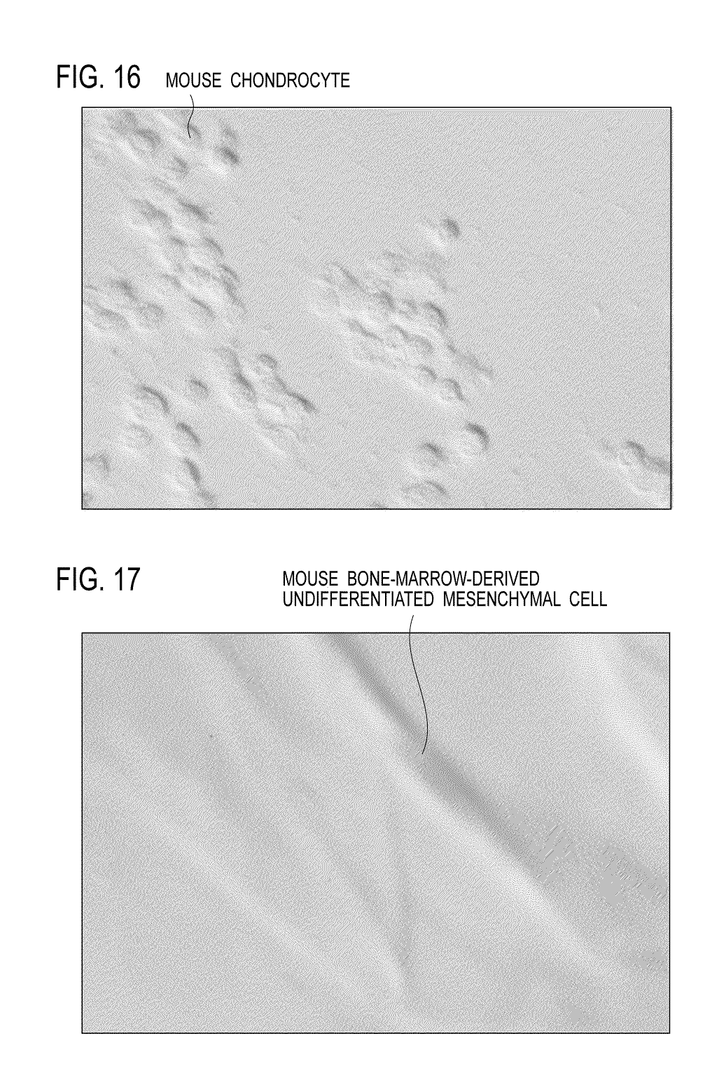

First, a cell culture membrane composed of a DNA photoproduct was sterilized with ethanol of 70% in volume concentration for 30 minutes. Next, a cell culture membrane composed of a photoproduct was set on a basal plane of well of a petri dish. After that, a well was filled with the same cell culture medium as that of a first embodiment, and was dispersed with mouse chondrocytes (ATDC5). After one day since mouse chondrocytes had been dispersed, as shown in FIG. 14, mouse chondrocytes were adhered on a cell culture membrane composed of a DNA photoproduct. As shown in FIG. 15, three days later, mouse chondrocytes were still adhered on a surface of cell culture membrane composed of a DNA photoproduct, and as shown in FIG. 16, seven days later, mouse chondrocytes were still adhered on a surface of cell culture membrane composed of a DNA photoproduct. Moreover, a cell culture membrane composed of a DNA photoproduct was not dissolved in a cell culture medium.

### Second Example of Second Embodiment

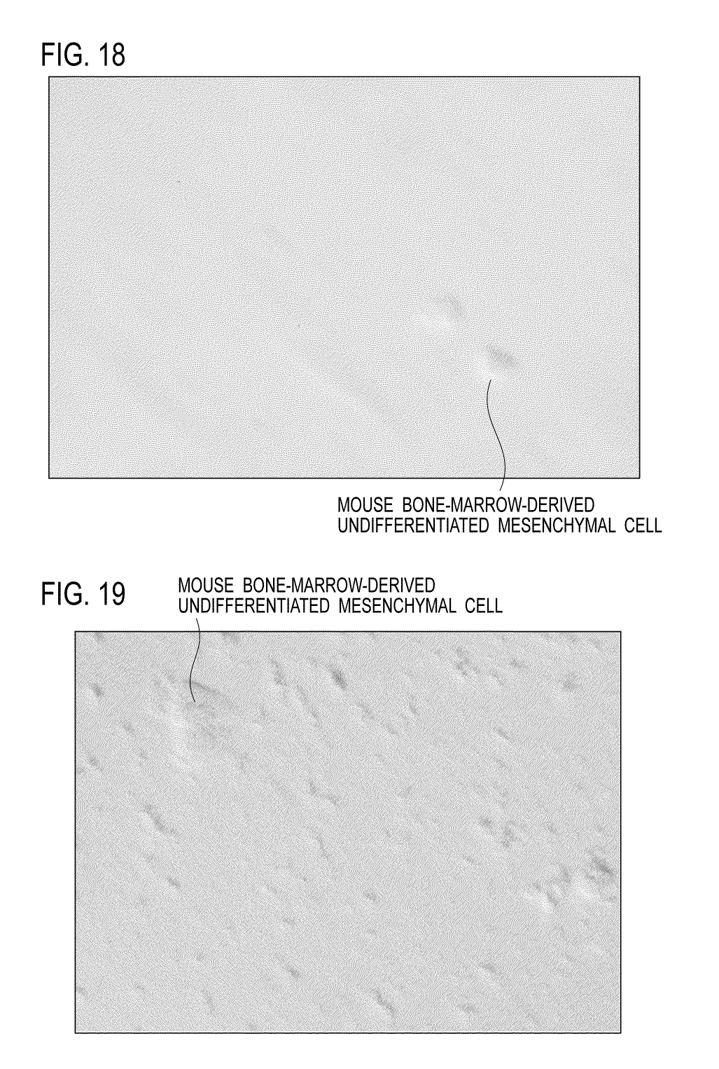

A sterilized cell culture membrane composed of a DNA photoproduct was set on a basal plane of well of a petri dish, a well was filled with the same cell culture medium as that of a first embodiment. After that, mouse bone-mallow-derived undifferentiated mesenchymal cells (C3H10T1/2) were dispersed. After one day since mouse bone-mallow-derived undifferentiated mesenchymal cells had been dispersed, as shown in FIG. 17, mouse bone-mallow-derived undifferentiated mesenchymal cells were adhered on a cell culture membrane composed of a DNA photoproduct. As shown in FIG. 18, three days later, mouse bone-mallow-derived undifferentiated mesenchymal cells were still adhered on a surface of cell culture membrane composed of a DNA photoproduct, and as shown in FIG. 19, seven days later, mouse bone-mallow-derived undifferentiated mesenchymal cells were still adhered on a surface of cell culture membrane composed of a DNA photoproduct. Moreover, a cell culture membrane composed of a DNA photoproduct was not dissolved in a cell culture medium.

### Third Embodiment

A cell culture membrane according to a third embodiment is composed of a salt of a mixture of DNA and salmon-derived type I collagen (Ihara Suisan Co. Ltd. made) and calcium ions or magnesium ions. A production method for a cell culture membrane according to a third embodiment will be explained. First, 1.5 g of sodium salt of DNA was dissolved in 200 ml of pure water, and an aqueous solution of sodium salt of DNA is prepared. Moreover, 5 g of salmon-derived type I collagen is dissolved in 100 ml of pure water, and an aqueous solution of salmon-derived type I collagen is prepared. Next, 40 ml of aqueous solution of sodium salt of DNA and 20 ml of aqueous solution of salmon-derived type I collagen are mixed together, and a mixed solution is prepared. After that, a mixed solution is dropped on a basal plane of fluorine resin petri dish of 100 mm in diameter and dried, then, an water-soluble film composed of a mixture of DNA and salmon-derived type I collagen is formed on a basal plane of petri dish. Next, by immersing an water-soluble film into, for example, 1 mol/l of CaCl₂ aqueous solution, sodium ions are substituted with calcium ions. As a result, a mixture of DNA and salmon-derived type I collagen is cross-linked with calcium ions, a cell culture membrane according to a third embodiment is formed. After that, a cell culture membrane is rinsed with ultrapure water, and dried.

### Modified Example of Third Embodiment

A cell culture membrane according to a modified example of a third embodiment is composed of a mixture of DNA ionically-cross-linked with calcium ions or sodium ions and salmon-derived type I collagen. A production method for a cell culture membrane according to a modified example of a third embodiment will be explained. First, in the same way as a first embodiment, DNA ionically-cross-linked with calcium ions or magnesium ions is formed. After a cross-linked DNA is rinsed with purified water, immersed for one night in a salmon-derived type I collagen aqueous solution of 5% in weight concentration, and a cell culture membrane composed of a mixture of DNA and salmon-derived type I collagen is formed. After that, salmon-derived type I collagen not contained in a free volume of ionically-cross-linked DNA is removed, and a cell culture membrane is dried.

### First Example of Third Embodiment

A cell culture membrane composed of a calcium salt of a mixture of DNA and salmon-derived type I collagen was set on basal planes of wells 111A and 111B of the petri dish 101 shown in FIG. 6, respectively. Note that, by preparing a mixture of DNA and salmon-derived type I collagen and ionically-cross-linking a mixture with calcium ions, a calcium salt of a mixture of DNA and salmon-derived type I collagen was produced. Moreover, a cell culture membrane composed of a mixture of DNA ionically-cross-linked with calcium ions and salmon-derived type I collagen was set on basal planes of wells 111C and 111D of the petri dish 101, respectively. Note that, after DNA was cross-linked with calcium ions, a mixture of ionically-cross-linked DNA with calcium ions and salmon-derived type I collagen was produced by mixing ionically-cross-linked DNA and salmon-derived type I collagen. After that, wells 111A to 111D were filled with the same cell culture medium as that of a first embodiment, mouse chondrocytes (ATDC5) were dispersed.

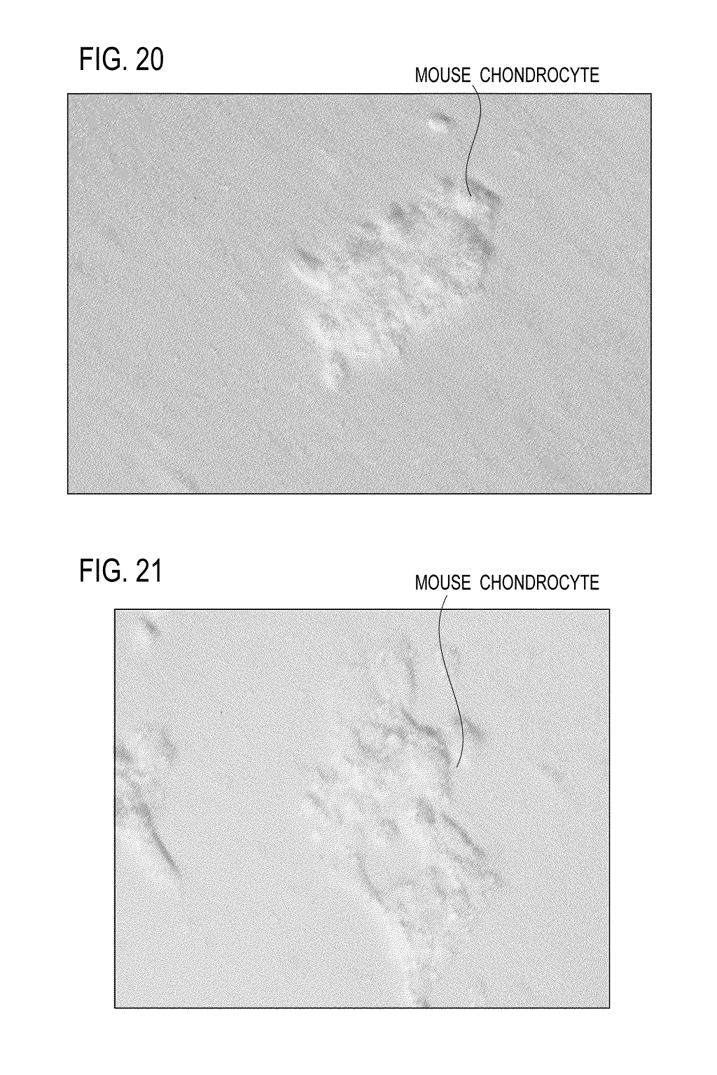

As shown in FIG. 20, mouse chondrocytes were adhered to a cell culture membrane composed of a calcium salt of a mixture of DNA and salmon-derived type I collagen, and increased. Note that, a photograph in FIG. 20 was taken after three days since mouse chondrocytes had been dispersed. A cell culture membrane composed of a calcium salt of a mixture of DNA and salmon-derived type I collagen was easily dissolved in a cell culture medium, compared with a cell culture membrane composed of a mixture of DNA ionically-cross-linked with calcium ions and salmon-derived type I collagen and a cell culture membrane composed of DNA ionically-cross-linked with calcium ions, and dissolved after three days. However, after a cell culture membrane of a calcium salt of a mixture composed of DNA and salmon-derived type I collagen had been dissolved, mouse chondrocytes were adhered on a basal plane of a petri dish and increased.

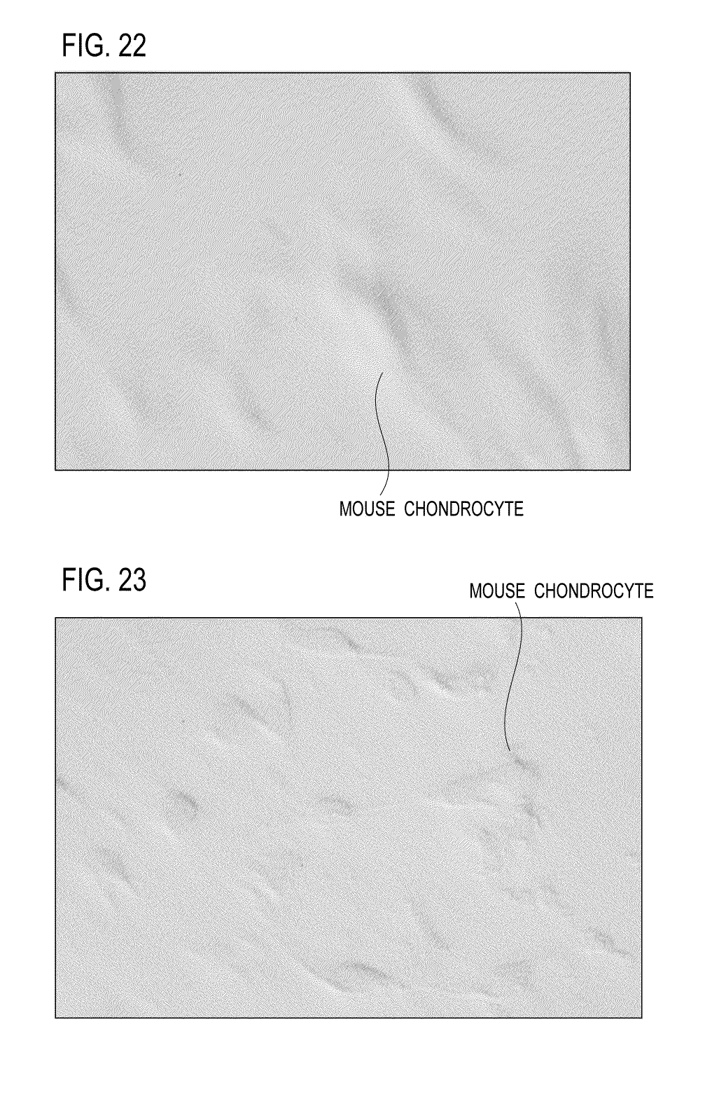

As shown in FIG. 21 and FIG. 22, mouse chondrocytes were also adhered to a cell culture membrane composed of a mixture of DNA ionically-cross-linked with calcium ions and salmon-derived type I collagen and increased. Note that, a photograph in FIG. 21 was taken after three days since mouse chondrocytes had been dispersed, and a photograph in FIG. 22 was taken after six days. Note that, a cell culture medium was substituted after four days since cells had been dispersed. Adhesiveness and multiplication property of a cell in a cell culture membrane composed of a mixture of DNA ionically-cross-linked with calcium ions and salmon-derived type I collagen were better than those in a cell culture membrane composed of a calcium salt of a mixture of DNA and salmon-derived type I collagen and a cell culture membrane composed of DNA ionically-cross-linked with calcium ions. Note that, although a cell culture membrane composed of a mixture of DNA ionically-cross-linked with calcium ions and salmon-derived type I collagen was easily dissolved, compared with a cell culture membrane composed only of DNA ionically-cross-linked with calcium ions, its form was preserved for five days or more since cells had been dispersed.

### Second Example of Third Embodiment

To check a first example of a third embodiment, a cell culture membrane composed of a mixture of DNA ionically-cross-linked with calcium ions and salmon-derived type I collagen was set on basal planes of wells 111A and 111B of the petri dish 101 shown in FIG. 6, respectively, and a cell culture membrane composed of a calcium salt of a mixture of DNA and salmon-derived type I collagen was set on basal planes of wells 111C and 111D, respectively. After that, the same cell culture medium as that of a first embodiment was filled in wells of 111A to 111D, and mouse chondrocytes were dispersed.

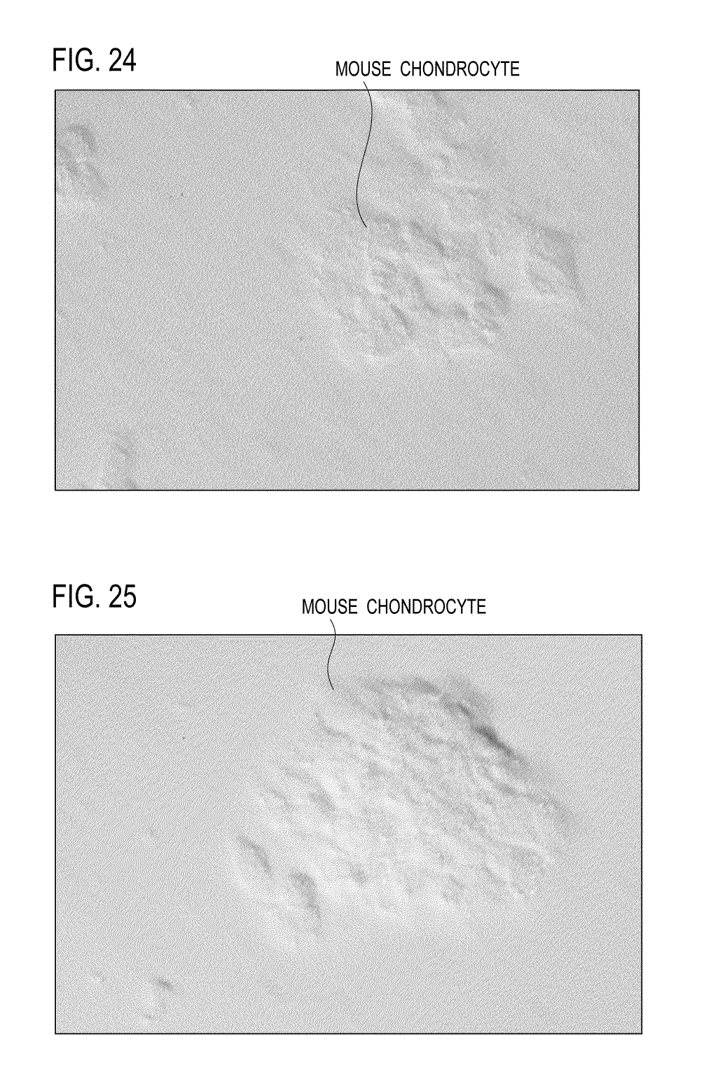

As shown in FIG. 23, FIG. 24, and FIG. 25, mouse chondrocytes were adhered on a cell culture membrane composed of a mixture of DNA ionically-cross-linked with calcium ions and salmon-derived type I collagen, and increased properly. Note that, a photograph in FIG. 23 was taken after one day since mouse chondrocytes had been dispersed, a photograph in FIG. 24 was taken after two days, and a photograph in FIG. 25 was taken after four days.

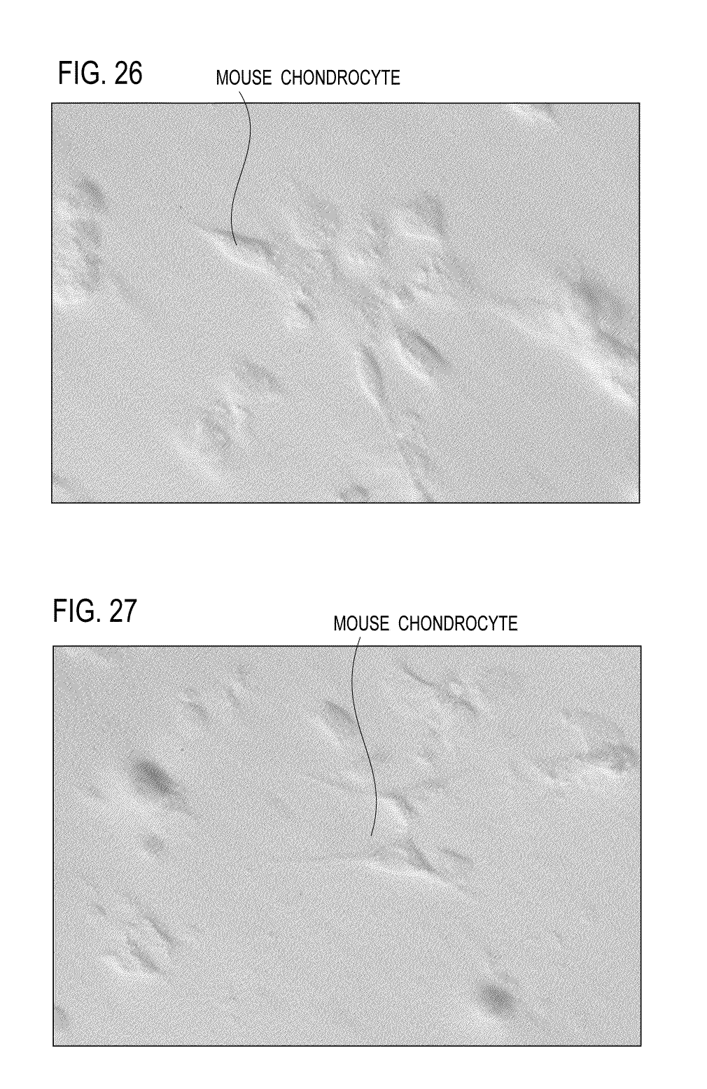

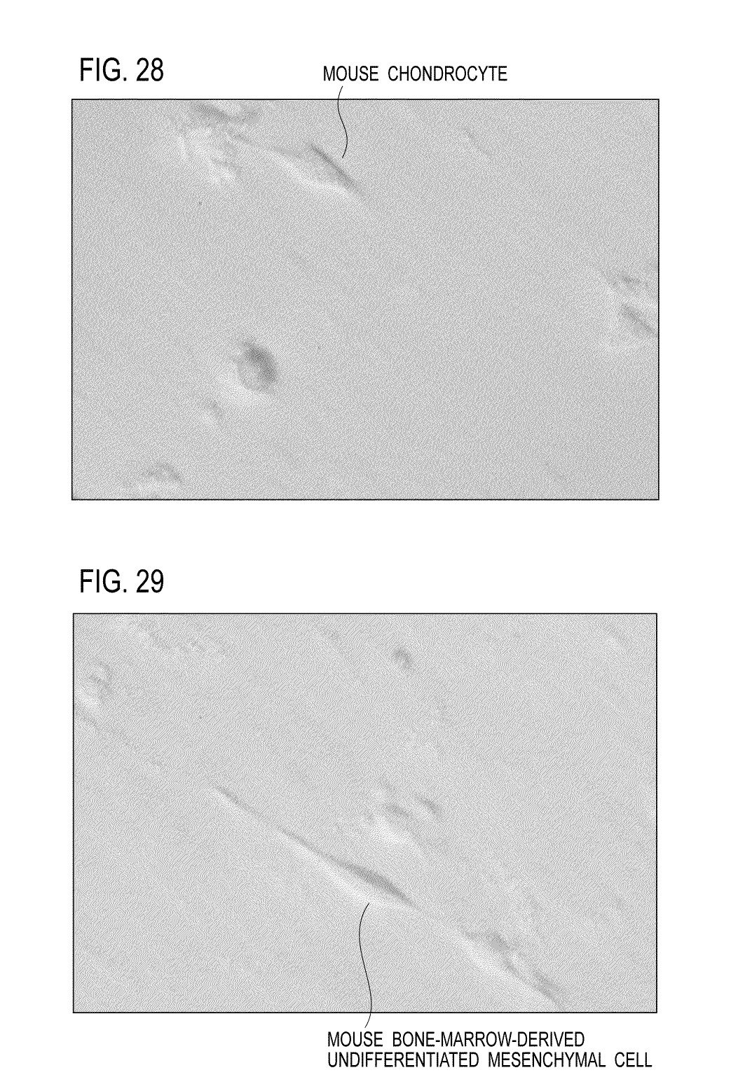

Moreover, as shown in FIG. 26, FIG. 27, and FIG. 28, mouse chondrocytes were adhered to a cell culture membrane composed of a calcium salt of a mixture of DNA and salmon-derived type I collagen. However, multiplication ability of mouse chondrocytes on a cell culture membrane composed of a calcium salt of a mixture of DNA and salmon-derived type I collagen was low, compared with that of mouse chondrocytes on a cell culture membrane composed of a mixture of DNA ionically-cross-linked with calcium ions and salmon-derived type I collagen. Note that, a photograph in FIG. 26 was taken after one day since mouse chondrocytes had been dispersed, a photograph in FIG. 27 was taken after two days, and a photograph in FIG. 28 was taken after four days.

In case of equal thickness, a soluble speed of a cell culture membrane composed of the mixture of DNA ionically-cross-linked with calcium ions and salmon-derived type I collagen was nearly equal to that of a cell culture membrane composed of a calcium salt of a mixture of DNA and salmon-derived type I collagen. Although the time until a cell culture membrane to be dissolved completely is proportional to a thickness, a form of cell culture membrane was preserved for two days without dissolution.

### Third Example of Third Embodiment

The same way as a first example of a third embodiment, a cell culture membrane composed of a calcium salt of a mixture of DNA and salmon-derived type I collagen and a cell culture membrane composed of a mixture of DNA ionically-cross-linked with calcium ions and salmon-derived type I collagen were set on basal planes of wells 111A to 111D of the petri dish 101 shown in FIG. 6. After that, the same cell culture medium as that of a first embodiment was filled in wells 111A to 111D, and mouse bone-mallow-derived undifferentiated mesenchymal cells (C3H10T1/2) were dispersed.

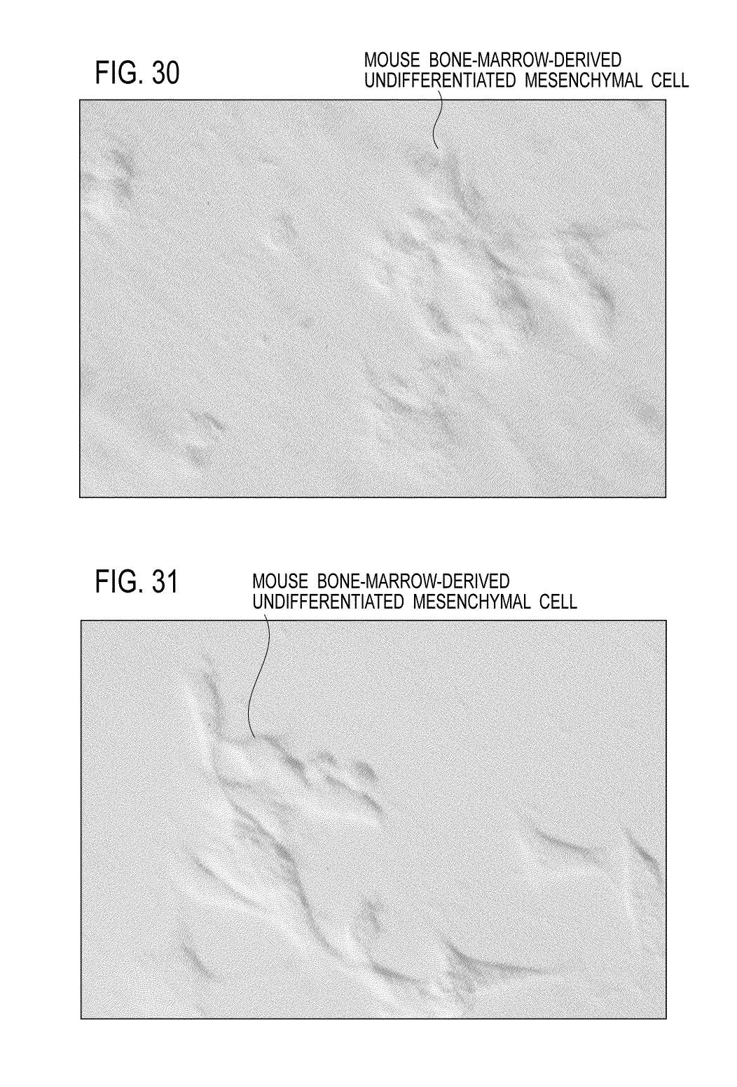

As shown in FIG. 29 and FIG. 30, mouse bone-mallow-derived undifferentiated mesenchymal cells were adhered to a cell culture membrane composed of a calcium salt of a mixture of DNA and salmon-derived type I collagen, and increased. Note that, a photograph in FIG. 29 was taken after one day since mouse bone-mallow-derived undifferentiated mesenchymal cells had been dispersed, a photograph in FIG. 30 was taken after two days since mouse bone-mallow-derived undifferentiated mesenchymal cells had been dispersed. A cell culture membrane composed of a calcium salt of a mixture of DNA and salmon-derived type I collagen was not dissolved until two days passed since mouse bone-mallow-derived undifferentiated mesenchymal cells had been dispersed.

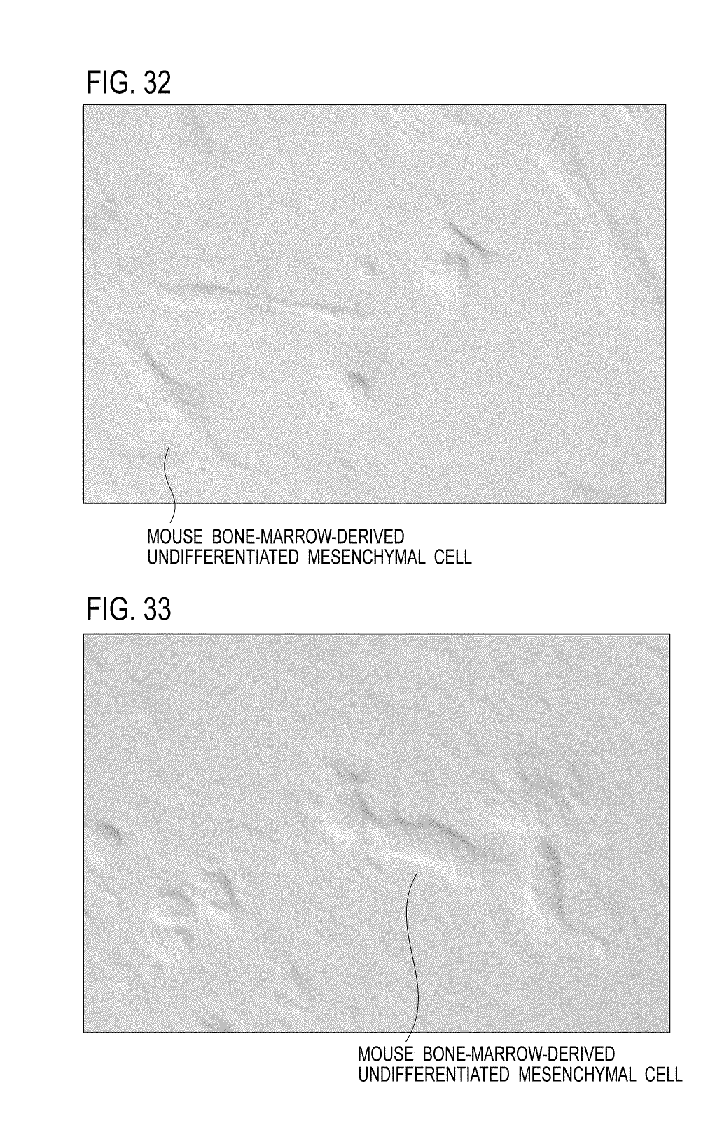

Moreover, as shown in FIG. 31 and FIG. 32, mouse bone-mallow-derived undifferentiated mesenchymal cells were also adhered to a cell culture membrane composed of a calcium salt of a mixture of DNA and salmon-derived type I collagen, and increased. Note that, a photograph in FIG. 31 was taken after one day since mouse bone-mallow-derived undifferentiated mesenchymal cells had been dispersed, a photograph in FIG. 32 was taken after two days since mouse bone-mallow-derived undifferentiated mesenchymal cells had been dispersed. A cell culture membrane composed of a calcium salt of a mixture of DNA and salmon-derived type I collagen was not dissolved until two days passed since mouse bone-mallow-derived undifferentiated mesenchymal cells had been dispersed.

### Fourth Example of Third Embodiment

To check a third example of a third embodiment, a cell culture membrane composed of a mixture of DNA ionically-cross-linked with calcium ions and salmon-derived type I collagen was set on basal planes of wells 111A and 111B of the petri dish 101 shown in FIG. 6, respectively, and a cell culture membrane composed of a calcium salt of a mixture of DNA and salmon-derived type I collagen was set on basal planes of wells 111C and 111D, respectively. After that, the same cell culture medium as that of a first embodiment was filled in wells 111A to 111D, and mouse bone-mallow-derived undifferentiated mesenchymal cells were dispersed.

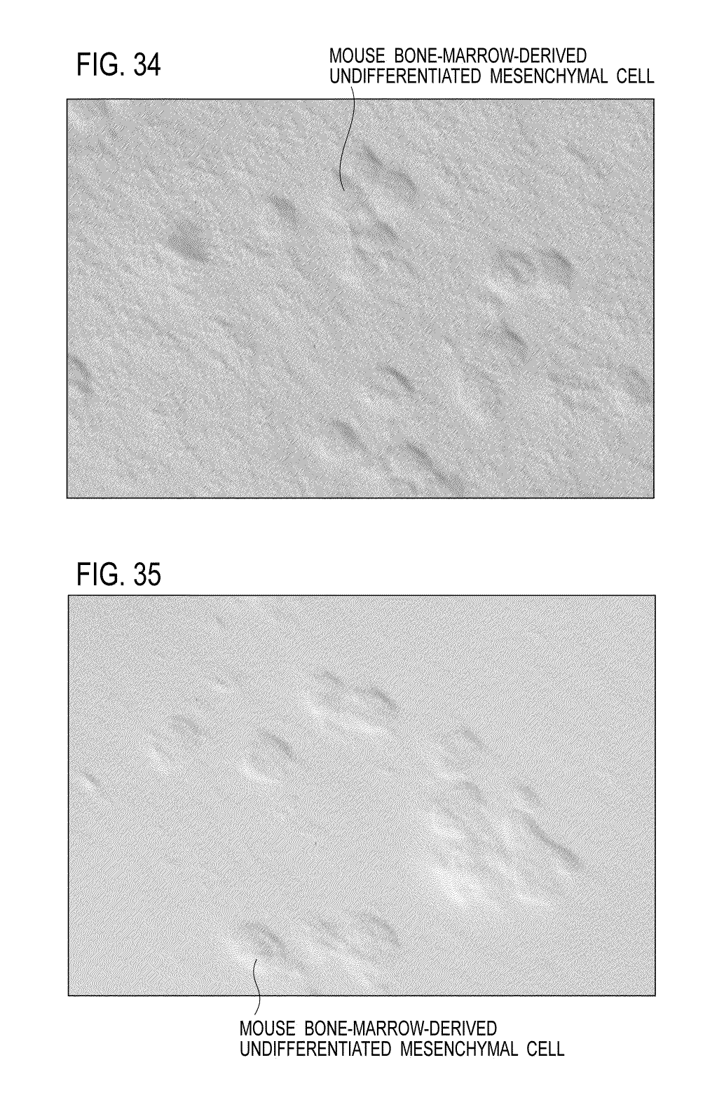

As shown in FIG. 33, FIG. 34, and FIG. 35, mouse bone-mallow-derived undifferentiated mesenchymal cells were adhered to a cell culture membrane composed of a mixture of DNA ionically-cross-linked with calcium ions and salmon-derived type I collagen, and increased. Note that, a photograph in FIG. 33 was taken after one day since mouse bone-mallow-derived undifferentiated mesenchymal cells had been dispersed, a photograph in FIG. 34 was taken after two days, and a photograph in FIG. 35 was taken after three days.

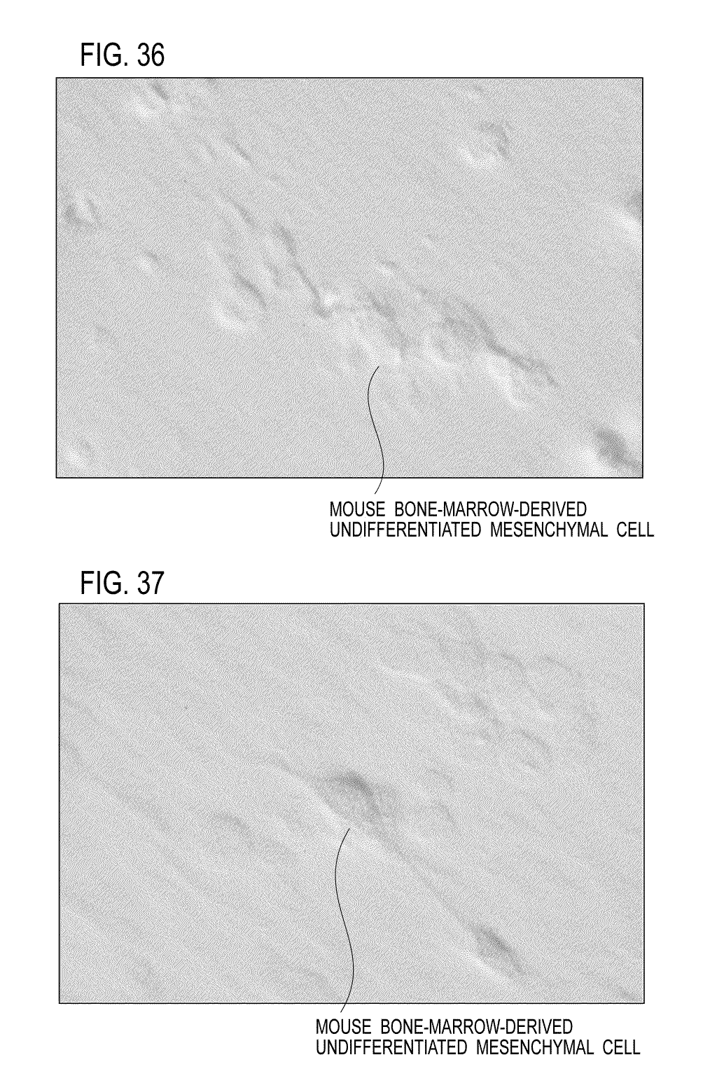

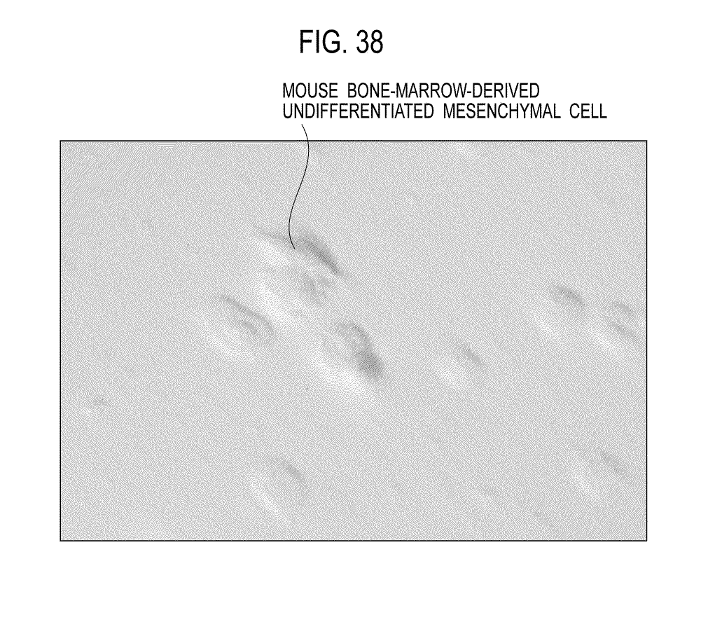

Moreover, as shown in FIG. 36, FIG. 37, and FIG. 38, mouse bone-mallow-derived undifferentiated mesenchymal cells were adhered to a cell culture membrane composed of a calcium salt of a mixture of DNA and salmon-derived type I collagen, and increased. Note that, a photograph was taken after one day since mouse bone-mallow-derived undifferentiated mesenchymal cells had been dispersed, a photograph in FIG. 37 was taken after two days, and a photograph in FIG. 38 was taken after three days.

A soluble speed of a cell culture membrane composed of a mixture of DNA ionically-cross-linked with calcium ions and salmon-derived type I collagen was nearly equal to that of a cell culture membrane composed of a salt of a mixture of DNA and salmon-derived type I collagen. Although the time until a cell culture membrane was dissolved completely is proportional to a thickness, a form of a cell culture membrane was preserved for at least two days without dissolution. Although a cell culture membrane begun to dissolve after two days later, a cell culture membrane was not dissolved completely until three days later.

### Fourth Embodiment

A cell culture membrane according to a fourth embodiment is composed of DNA ionically-cross linked with quaternary ammonium ions having long-chain alkyl groups shown in chemical formula (1). Note that, when a carbon number in an alkyl group becomes 13 or more, a quaternary ammonium ion shown in chemical formula (1) has cell toxicity. Hence, a carbon number in an alkyl group is preferable to be 6 to 12.

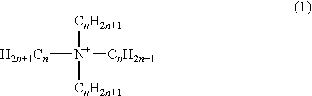

A cell culture membrane composed of DNA ionically-cross-linked with quaternary ammonium ions is transparent, and suitable for a cell observation with an optical microscopy. Moreover, it has biocompatibility and high affinity for a cell. Furthermore, it is not dissolved in a cell culture medium for two days or more, and its form can be preserved.

Next, a production method for a cell culture membrane according to a fourth embodiment will be explained. First, in the same way as a production method for a cell culture membrane according to a first embodiment, an water-soluble film composed of a sodium salt of DNA is formed. Then, an aqueous solution containing quaternary ammonium ions is dropped on a petri dish, and an water-soluble film is immersed in an aqueous solution containing quaternary ammonium ions for one day at room temperature. By immersing an water-soluble film in an aqueous solution containing quaternary ammonium ions, sodium ions are substituted with quaternary ammonium ions, and DNA is ionically-cross-linked with quaternary ammonium ions. A gelled DNA ionically-cross-linked with quaternary ammonium ions is collected as a cell culture membrane according to a forth embodiment.

### Example of Forth Embodiment

A cell toxicity test of a cell culture membrane ionically-cross-linked with quaternary ammonium ions was done. A cell toxicity test was followed a separate attachment [Fundamental idea of biological safety evaluation for medical instruments] in [Fundamental idea of biological safety test necessary for approval application of manufacturing medical devices (in port)] (Pharmaceutical and Medical Safety Bureau Ministry of Hearth notification No. 0213001, 2003). Concretely, a cell culture membrane shaped as a semicircular film was set so as to occupy on a half space of circular basal plane of a petri dish. Next, a check was done whether V79 cells increased on both of basal plane of a petri dish to form a colony or not. As a result, V79 cells increased on a cell culture membrane composed of DNA ionically-cross-linked with quaternary ammonium ions having a carbon number of 6 to 12 in an alkyl group, and colonies were formed. However, V79 cells did not increased on a cell culture membrane composed of DNA ionically-cross-linked with quaternary ammonium ions having a carbon number of 13 or more in an alkyl group.

### Fifth Embodiment

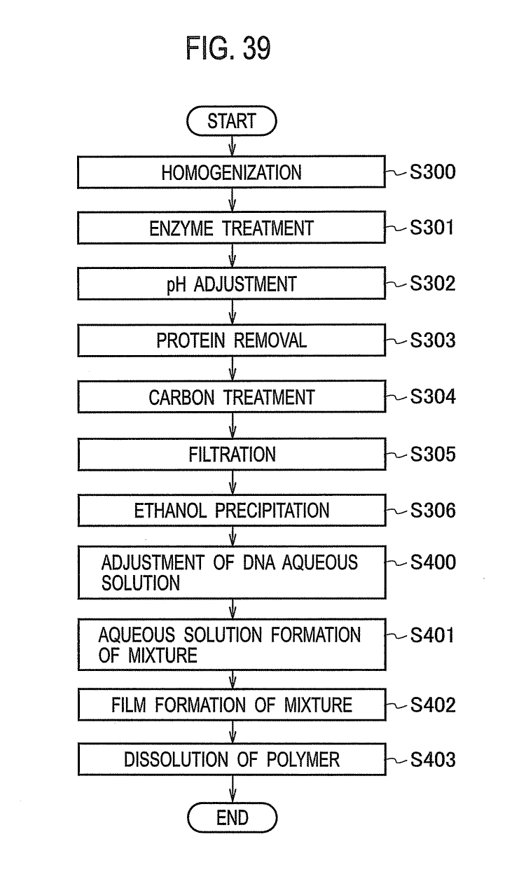

A porous material according to a fifth embodiment is composed of deoxyribonucleic acid (DNA) and is prepared fine pores of 1 nm to 100 nm in diameter. Note that, a fine pore diameter is a value measured by a scanning electron microscopy (SEM). Next, using a flow chart shown in FIG. 39, a production method for a porous material according to a fifth embodiment will be explained.

(a) At a step S300, salmon milt, mid-gut gland of scallop or the like is homogenized with a homogenizer. Next, salmon milt or mid-gut gland of scallop homogenized is filtrated, and a first filtrate is obtained. At a step S301, a protein proteolytic enzyme e.g. protease and the like is added to a first filtrate, proteins contained in a first filtrate are degraded. Then, at a step S302, pH of a first filtrate is adjusted, and at a step S303, proteins contained in a first filtrate are removed.

(b) At a step S304, a first filtrate is carbon-treated. At a step S305, a first filtrate is filtrated, and a second filtrate is obtained. At a step S306, ethanol is added to a second filtrate, a sodium salt of DNA which is a salt of purified DNA and monovalent cations is extracted. Then, a sodium salt of DNA is dried. Note that, DNA may be either a single strand or a double strand. Molecular weight of DNA is, for example, 100 kDa to 10,000 kDa. In the following example, highly-pure DNA having an average molecular weight of 6,600 kDa and a purity of 90% or more is used. Note that, omitting a step 300 to a step 306, DNA in the marketplace (Nihon Kagaku Shiryo Co. Ltd. made) may be used.

(c) At a step S400, 1 g of a sodium salt of DNA is dissolved in 100 ml of ultrapure water, and a DNA aqueous solution is prepared. At a step 401, stirring a DNA aqueous solution at 20° C., 1 g of poly(N-isopropyl acrylamide)(PNIPAAm) of a molecular weight of 200 kDa as an alcohol soluble polymer is added to a DNA aqueous solution, PNIPAAm is dissolved in a DNA aqueous solution, and an aqueous solution of a mixture of DNA and PNIPAAm is obtained.

(d) At a step 402, an aqueous solution of a mixture of DNA and PNIPAAm is poured on a plane e.g. a basal plane of vitreous petri dish coated with fluorine resin and the like. Next, by vacuum drying an aqueous solution of a mixture at room temperature, a transparent film composed of a mixture of DNA and PNIPAAm is formed on a basal plane of petri dish. In this film, a microphase-separated structure is formed between DNA and PNIPAAm.

(e) At a step 403, a film composed of a mixture of DNA and PNIPAAm is set in 100 ml of ethanol, and then, it is left for one day while ethanol is gently stirred. In the meantime, although DNA is insoluble to ethanol, PNIPAAm is soluble to ethanol. Therefore, by a nonsolvent-induced phase separation (NIPS) method, PNIPAAm in a mixture is dissolved and a porous material of white and opaque DNA is formed.

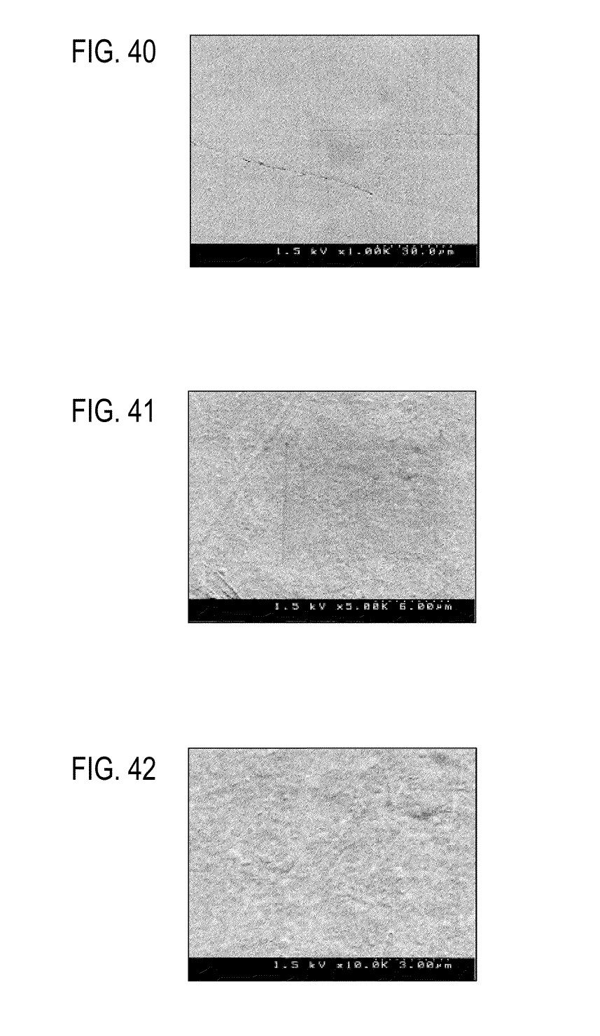

FIG. 40 shows a photograph of a film composed of a mixture of DNA and PNIPAAm obtained at a step 402 observed with an electron microscope at 1000-fold magnification. FIG. 41 shows a photograph of a film composed of a mixture of DNA and PNIPAAm observed with an electron microscope at 5000-fold magnification. FIG. 42 shows a photograph of a film composed of a mixture of DNA and PNIPAAm observed with an electron microscope at 10000-fold magnification. As shown in FIG. 40 to FIG. 42, fine pores were not observed on a surface of a film composed of a mixture of DNA and PNIPAAm.

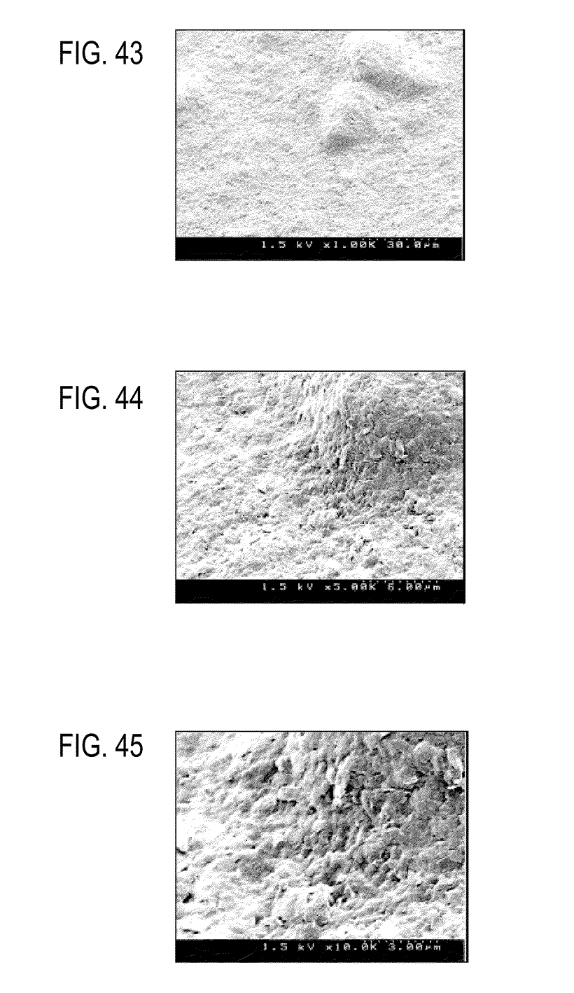

On the other hand, FIG. 43 shows a photograph of a porous material composed of DNA obtained at a step 403 observed with an electron microscope at 1000-fold magnification. FIG. 44 shows a photograph of a porous material composed of DNA observed with an electron microscope at 5000-fold magnification. FIG. 45 shows a photograph of a porous material composed of DNA observed with an electron microscope at 10000-fold magnification. As shown in FIG. 43 to FIG. 45, fine pores were observed on a surface of a porous material composed of DNA.

### Modified Example of Fifth Embodiment

In a fifth embodiment, although PNIPAAm was used as an alcohol soluble polymer, a polymer is not limited to it. Moreover, a polymer may also be either a natural polymer or a synthetic polymer. For example, poly(N-vinyl pyrrolidone) having a molecular weight of 150 kDa may be used as an alcohol soluble polymer.

Or poly(N-alkyl acrylamide) of an acrylamide derivative polymer or poly(N,N-dialkyl acrylamide) of a N,N-dialkyl acrylamide derivative polymer having an alkyl group of methyl, ethyl, propyl, isopropyl, butyl, isobutyl and the like, an alicyclic group of cyclohexyl, cyclonaphthyl and the like can be used as an alcohol soluble polymer.

Or poly(N-vinyl lactam) having a lactam ring as a side chain, e.g., poly(N-vinyl pyrrolidone), poly(N-vinyl caprolactam), poly(N-vinyl laurolactam) and the like, or poly(alkyl vinyl ether) e.g. poly(methyl vinyl ether), poly(ethyl vinyl ether) and the like can also be used as an alcohol soluble polymer.

Or an acrylate derivative polymer e.g. poly(hydroxylethyl acrylate), poly(hydroxyl ethyl methacrylate) and the like, or a cellulose derivative e.g. carboxymethyl cellulose, carboxyethyl cellulose, methyl alginate and the like, or other polymers soluble to DNA and forming a microphase-separated structure, can also be used as an alcohol soluble polymer.

### First Example of Fifth Embodiment

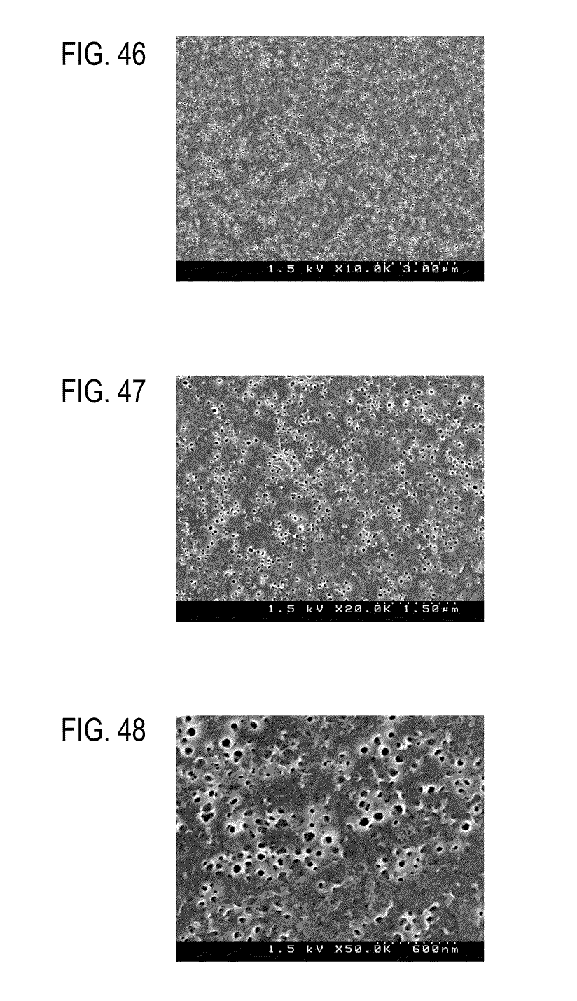

Using DNA of 9.2 mmol/l concentration and PNIPAAm of 20 mmol/l concentration, a porous material according to a fifth example of a fifth embodiment was produced. In the following description, aback side of porous material refers to a side contacted with a basal plane of petri dish in production, and a side not contacted with a basal plane to a surface of porous material. FIG. 46 is a surface image of a porous material observed with SEM at 10,000-fold magnification, FIG. 47 is a surface image of a porous material observed at 20,000-fold magnification, and FIG. 48 is a surface image of a porous material observed at 50,000-fold magnification. By analyzing surface images in FIG. 46 to FIG. 48 using an image analysis software (Image-J), a fine pore diameter produced in a porous material was found to be 20 nm to 80 nm. Moreover, a porosity defined by a ratio of total opening area of a plural of fine pores to a surface area was 1.9%.

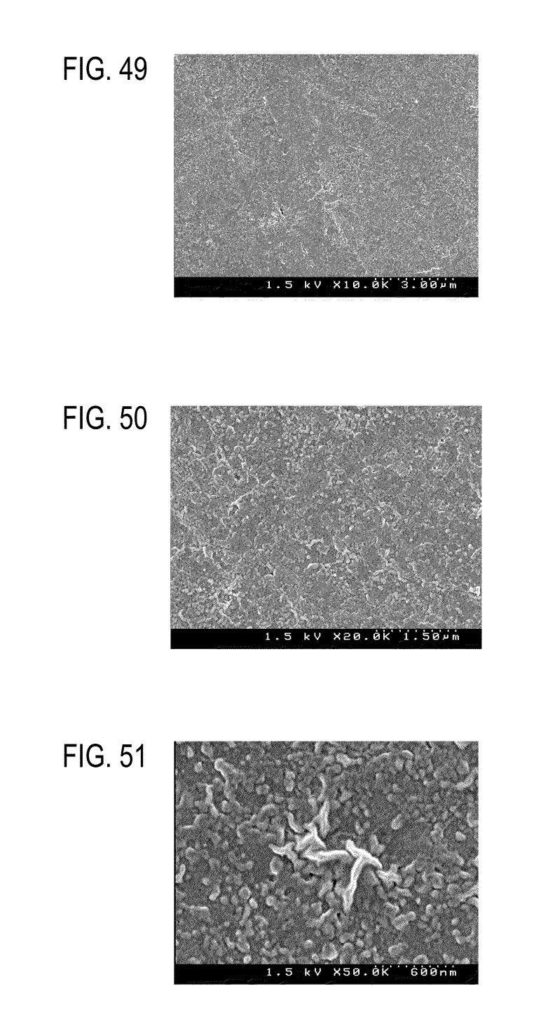

FIG. 49 is a back side image of a porous material observed at 10,000-fold magnification, FIG. 50 is a back side image of a porous material observed at 20,000-fold magnification and FIG. 51 is a back side image of a porous material observed at 50,000-fold magnification. In a back side, fine pores were scarcely observed.

### Second Example of Fifth Embodiment

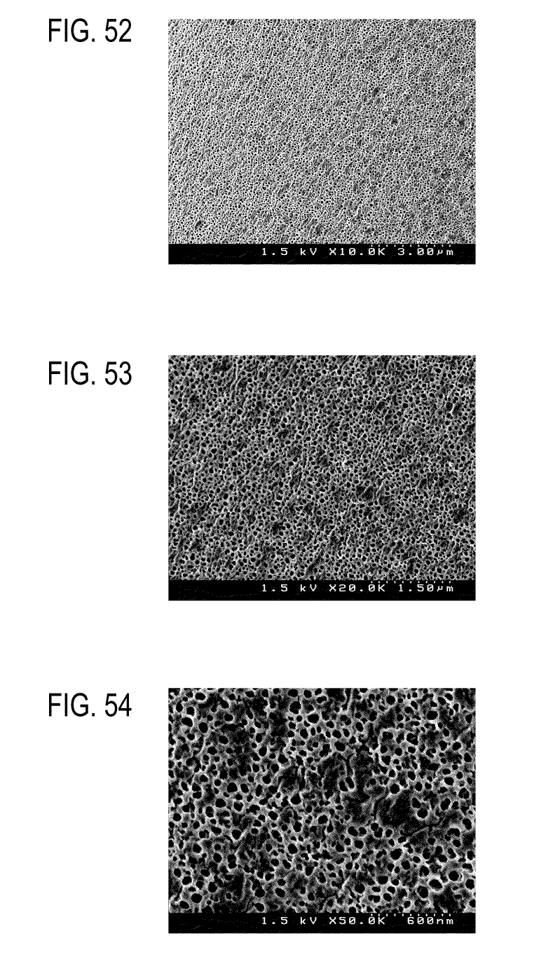

Using DNA of 8 mmol/l concentration and PNIPAAm of 50 mmol/l concentration, a porous material according to a second example of a fifth embodiment was produced. FIG. 52 is a surface image of a porous material observed with SEM at 10,000-fold magnification, FIG. 53 is a surface image of a porous material observed at 20,000-fold magnification, and FIG. 54 is a surface image of a porous material observed at 50,000-fold magnification. By analyzing surface images in FIG. 52 to FIG. 54 using an image analysis software, a fine pore diameter prepared in a porous material was found to be 20 nm to 80 nm. Moreover, a porosity defined by a ratio of total opening area of a plural of fine pores to a surface area was 8.7%. Hence, compared with FIG. 46 to FIG. 48, it was shown that a porosity was increased by increasing PNIPAA concentration.

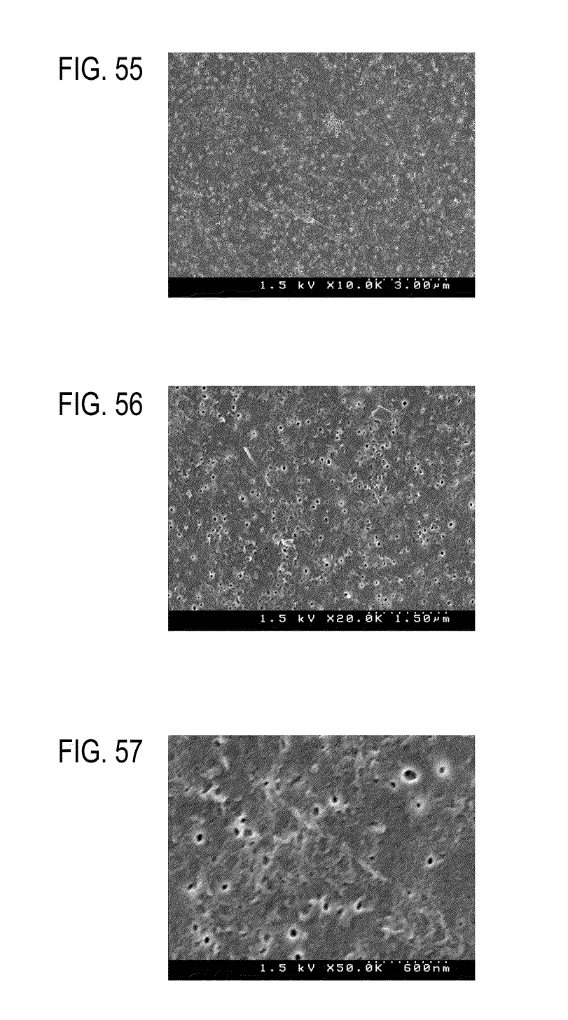

FIG. 55 is a back side image of a porous material observed at 10,000-fold magnification, FIG. 56 is a back side image of a porous material observed at 20,000-fold magnification, and FIG. 57 is a back side image of a porous material observed at 50,000-fold magnification. Fine pores were also observed on a back side, though the number of fine pores was less than that on a surface.

### Third Example of Fifth Embodiment

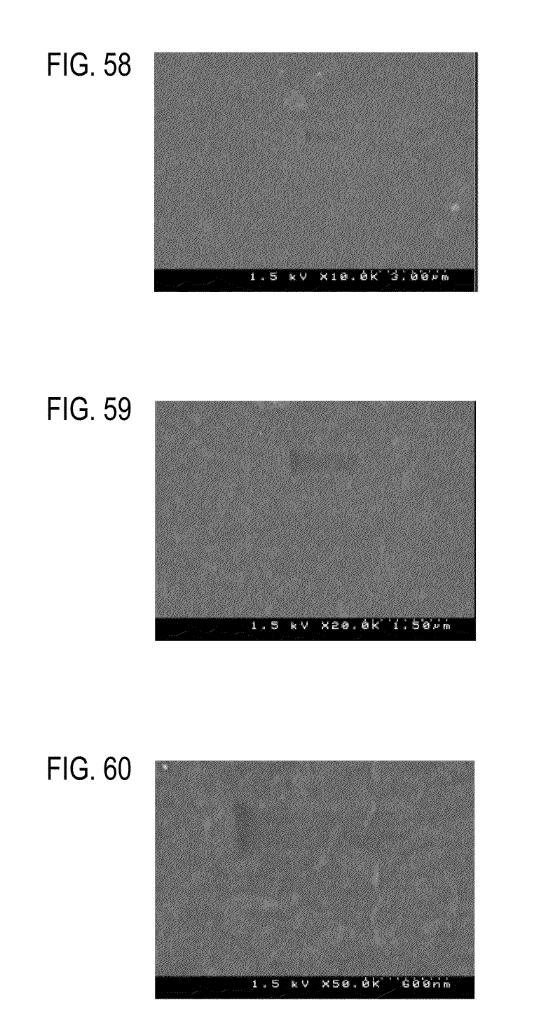

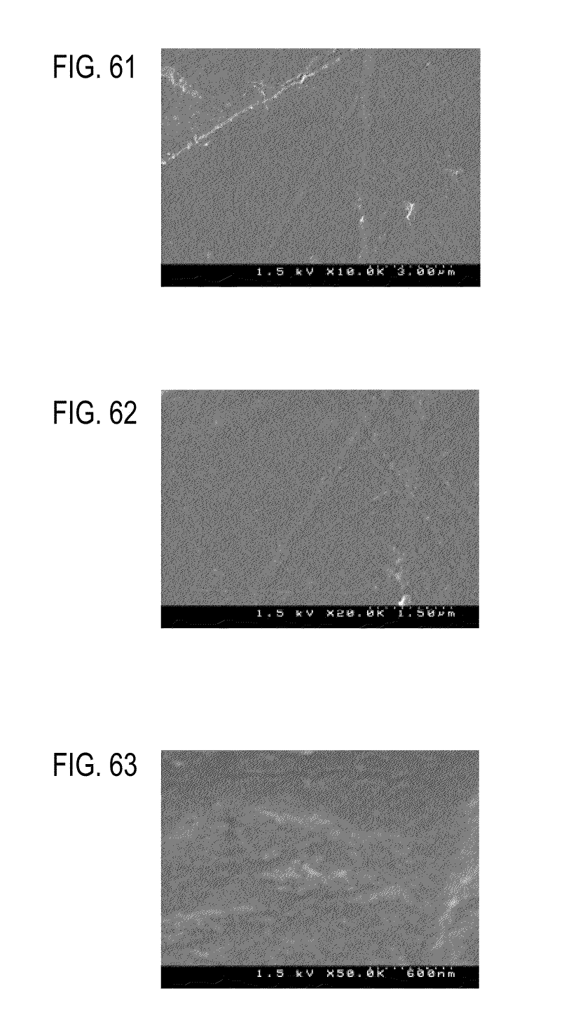

By mixing DNA of 10 mmol/l concentration and PNIPAAm of 5 mmol/l concentration, a film was produced. FIG. 58 is a surface image of a film composed of a mixture of DNA and PNIPAAm observed with SEM at 10,000-fold magnification, FIG. 59 is a surface image of a film composed of a mixture of DNA and PNIPAAm observed at 20,000-fold magnification, and FIG. 60 is a surface image of a film composed of a mixture of DNA and PNIPAAm observed at 50,000-fold magnification. Moreover, FIG. 61 is a back side image of a film composed of a mixture of DNA and PNIPAAm observed with SEM at 10,000-fold magnification, FIG. 62 is a back side image of a film composed of a mixture of DNA and PNIPAAm observed at 20,000-fold magnification, and FIG. 63 is a back side image of a film composed of a mixture of DNA and PNIPAAm observed at 50,000-fold magnification. In any magnification, fine pores were not observed on both of a surface and a back side.

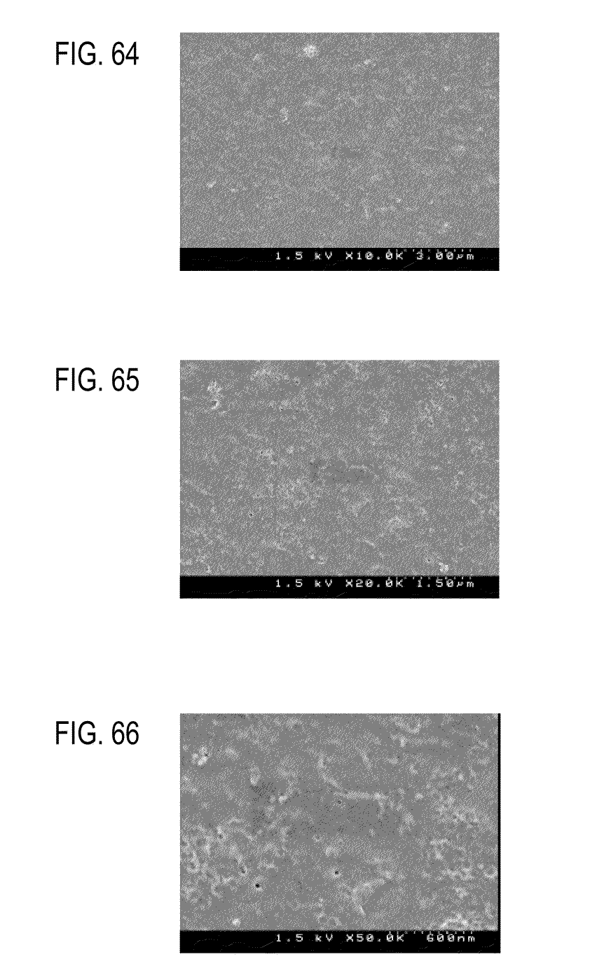

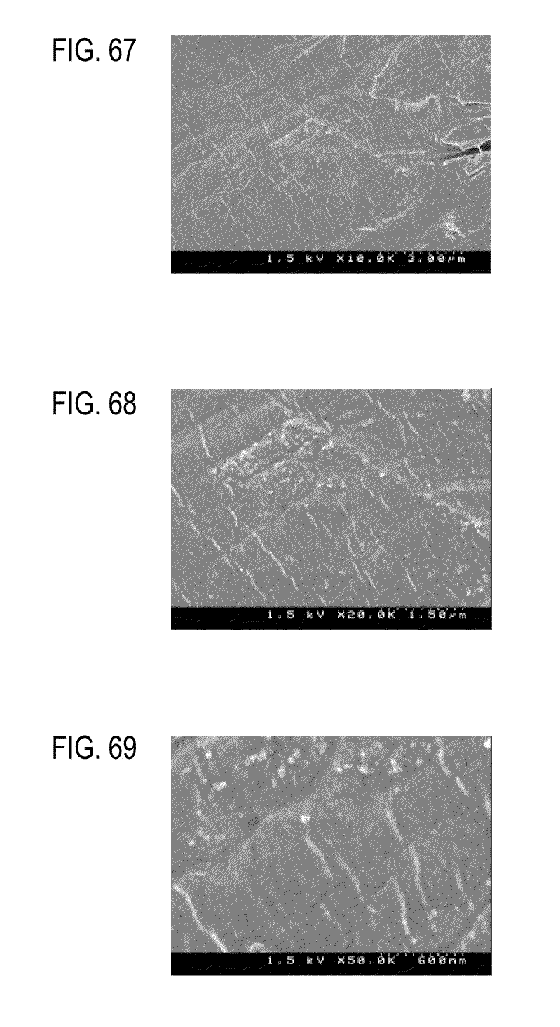

Next, a film composed of a mixture of DNA and PNIPAAm was set in ethanol, PNIPAAm was dissolved, and a porous material according to a third example of a fifth embodiment was produced. FIG. 64 is a surface image of a porous material observed at 10,000-fold magnification, FIG. 65 is a surface image of a porous material observed at 20,000-fold magnification, and FIG. 66 is a surface image of a porous material observed at 50,000-fold magnification. A surface became rough with an ethanol treatment, and fine pores were observed. FIG. 67 is a back side image of a porous material observed at 10,000-fold magnification, FIG. 68 is a back side image of a porous material observed at 20,000-fold magnification, and FIG. 69 is a back side image of a porous material observed at 50,000-fold magnification. On a back side, fine pores were not observed.

### Fourth Example of Fifth Embodiment

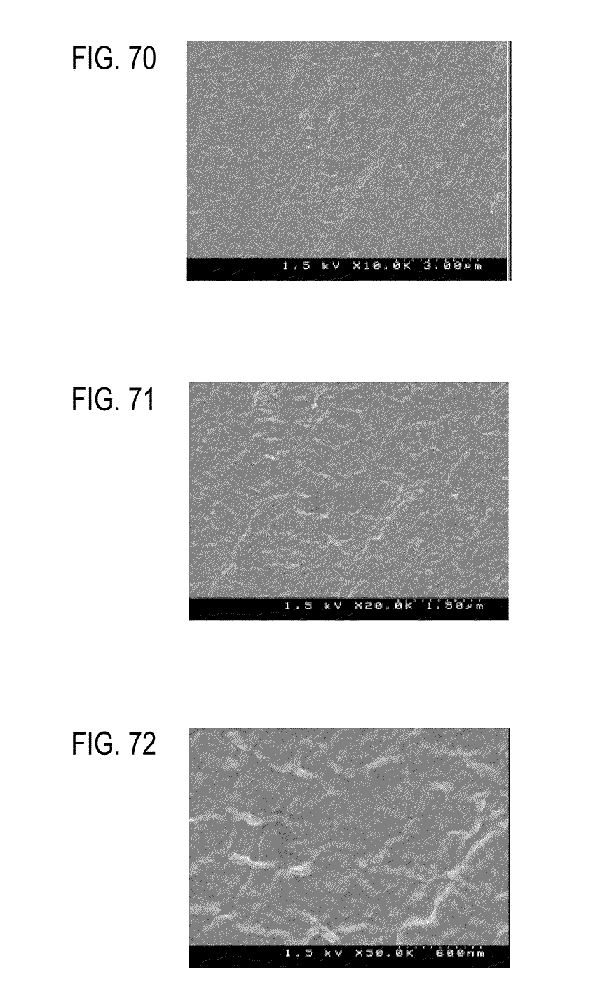

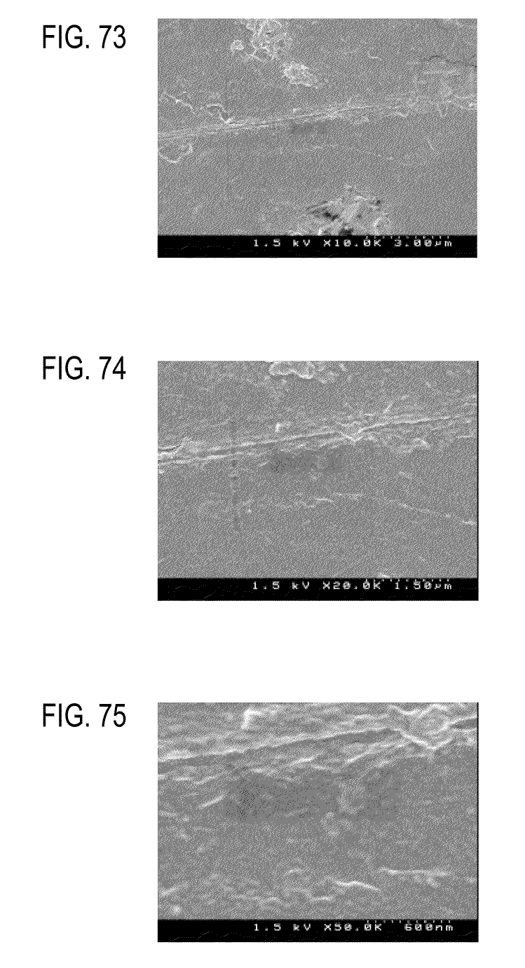

By mixing DNA of 10 mmol/l concentration and PNIPAAm of 10 mmol/l concentration, a film was produced. FIG. 70 is a surface image of a film composed of a mixture of DNA and PNIPAAm observed with SEM at 10,000-fold magnification, FIG. 71 is a surface image of a film composed of a mixture observed at 20,000-fold magnification, and FIG. 72 is a surface image of a film composed of a mixture observed at 50,000-fold magnification. Moreover, FIG. 73 is a back side image of a film composed of a mixture of DNA and PNIPAAm observed with SEM at 10,000-fold magnification, FIG. 74 is a back side image of a film composed of a mixture observed at 20,000-fold magnification, and FIG. 75 is a back side image of a film composed of a mixture observed at 50,000-fold magnification. In any magnification, fine pores were not observed on both a surface and a back side.

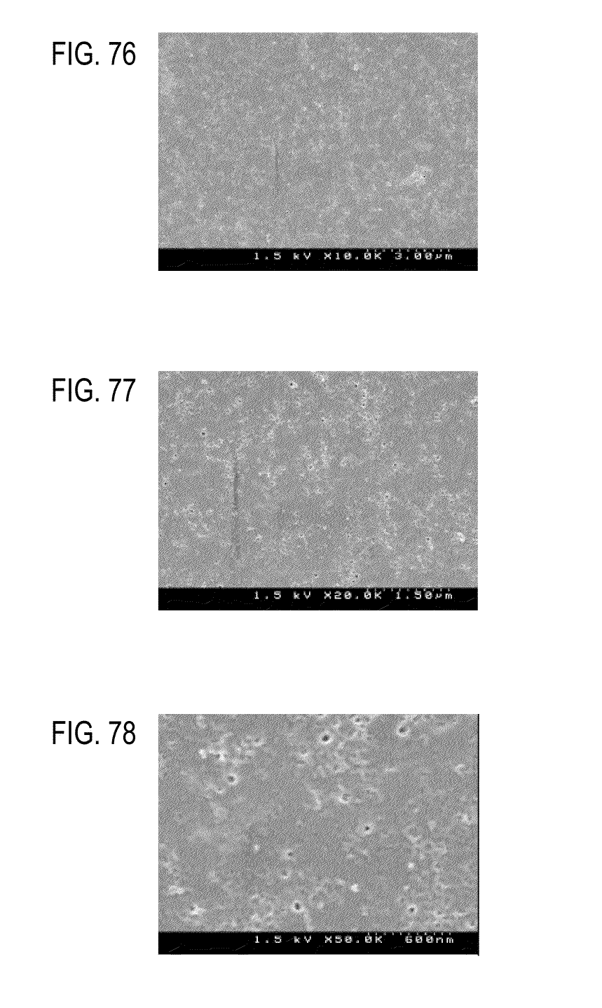

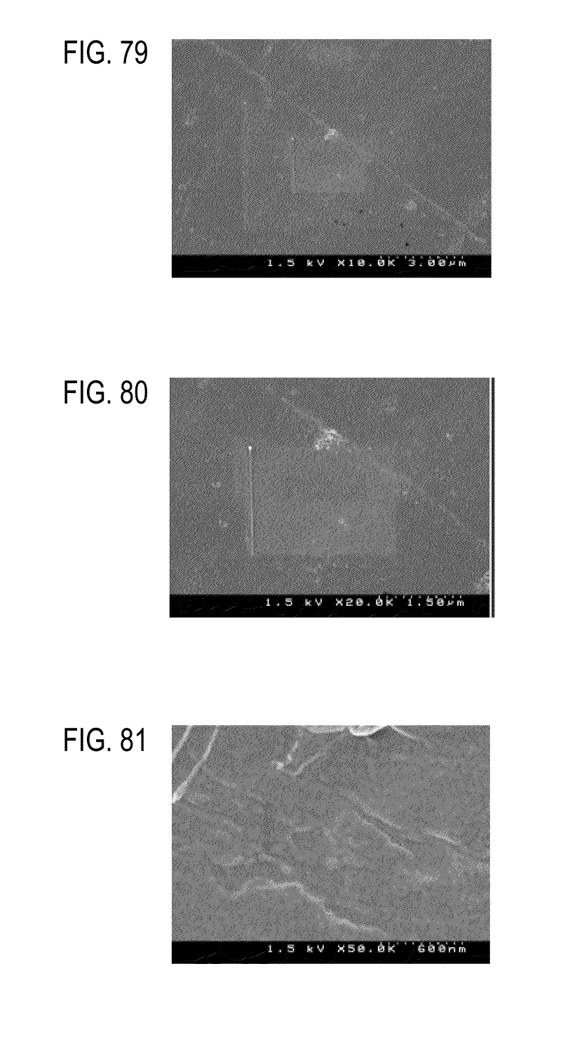

Next, a film composed of a mixture of DNA and PNIPAAm was set in ethanol, PNIPAAm was dissolved, and a porous material according to a fourth example of a fifth embodiment was produced. FIG. 76 is a surface image of a porous material observed at 10,000-fold magnification, FIG. 77 is a surface image of a porous material observed at 20,000-fold magnification, and FIG. 78 is a surface image of a porous material observed at 50,000 magnification. A surface became rough with an ethanol treatment, and fine pores were observed. FIG. 79 is a back side image of a porous material observed at 10,000-fold magnification, FIG. 80 is a back side image of a porous material observed at 20,000-fold magnification, and FIG. 81 is a back side image of a porous material observed at 50,000-fold magnification. Fine pores were also observed on a back side, though the number of fine pores was less than that on a surface.

### Fifth Example of Fifth Embodiment

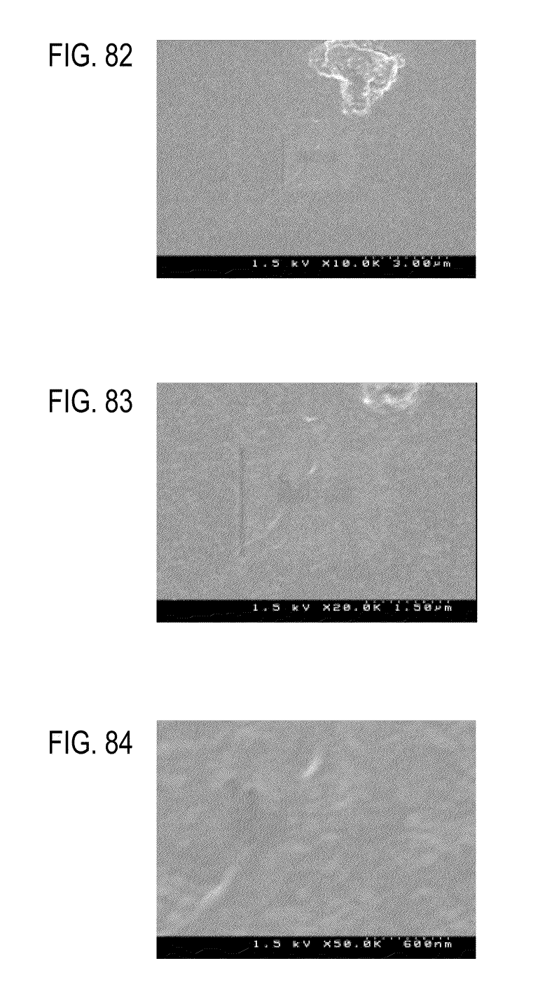

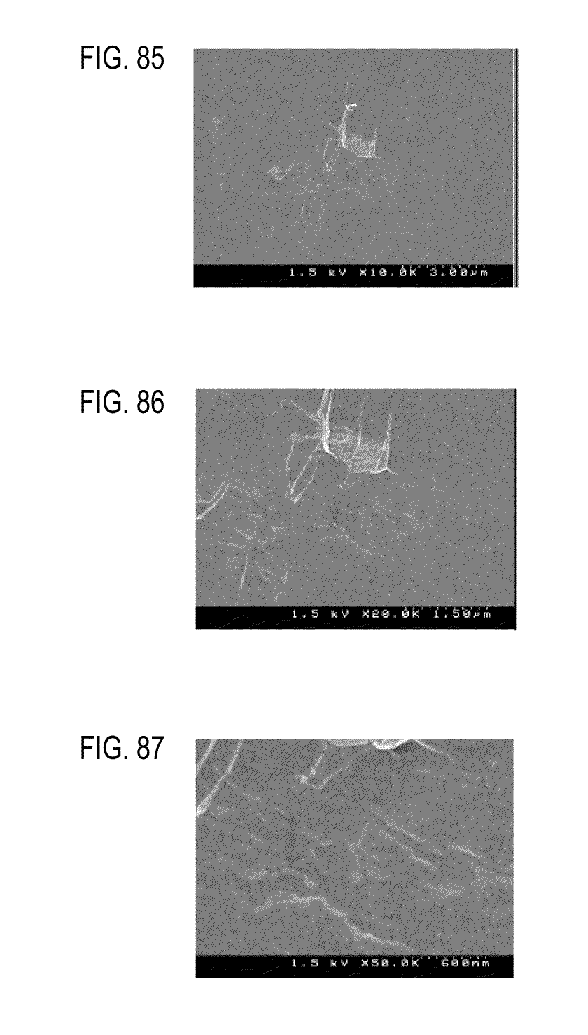

By mixing DNA of 10 mmol/l concentration and PNIPAAm of 20 mmol/l concentration, a film was produced. FIG. 82 is a surface image of a film composed of a mixture of DNA and PNIPAAm observed with SEM at 10,000-fold magnification, FIG. 83 is a surface image of a film composed of a mixture observed at 20,000-fold magnification, and FIG. 84 is a surface image of a film composed of a mixture observed at 50,000-fold magnification. Moreover, FIG. 85 is a back side image of a film composed of a mixture of DNA and PNIPAAm observed with SEM at 10,000-fold magnification, FIG. 86 is a back side image of a film composed of a mixture observed at 20,000-fold magnification, and FIG. 87 is a back side image of a film composed of a mixture observed at 50,000-fold magnification. In any magnification, fine pores were not observed on both a surface and a back side.

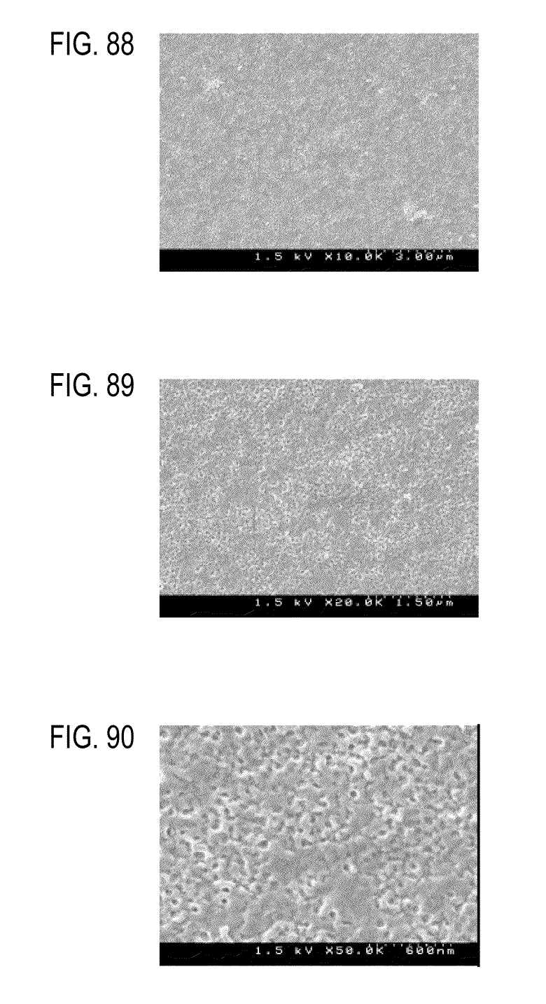

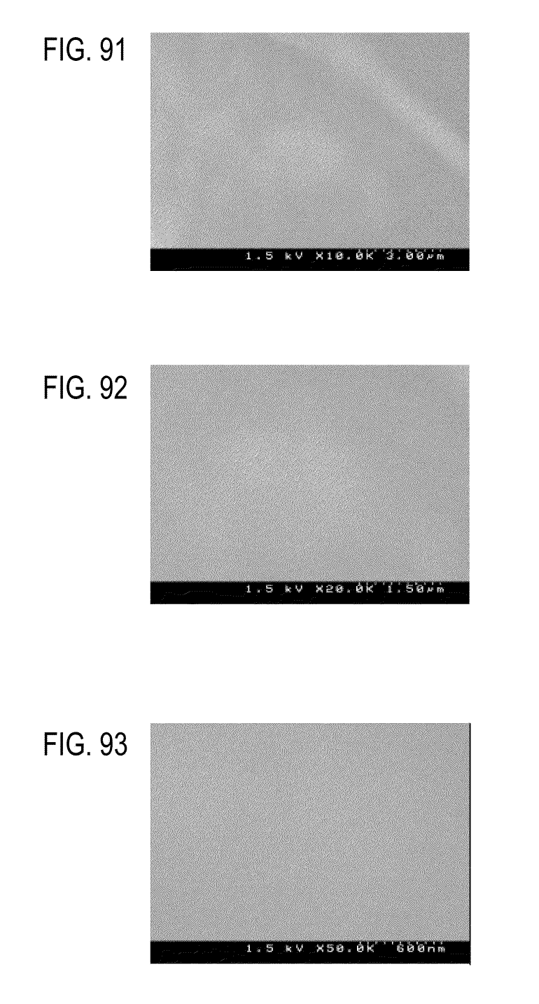

Next, a film composed of a mixture of DNA and PNIPAAm was set in ethanol, PNIPAAm was dissolved, and a porous material according to a fifth example of a fifth embodiment was produced. FIG. 88 is a surface image of a porous material observed at 10,000-fold magnification, FIG. 89 is a surface image of a porous material observed at 20,000-fold magnification, and FIG. 90 is a surface image of a porous material observed at 50,000-fold magnification. A surface became rough with an ethanol treatment, and fine pores were observed. FIG. 91 is a back side image of a porous material observed at 10,000-fold magnification, FIG. 92 is a back side image of a porous material observed at 20,000-fold magnification, and FIG. 93 is a back side image of a porous material observed at 50,000-fold magnification. On a back side, fine pores were not observed.

### Sixth Example of Fifth Embodiment

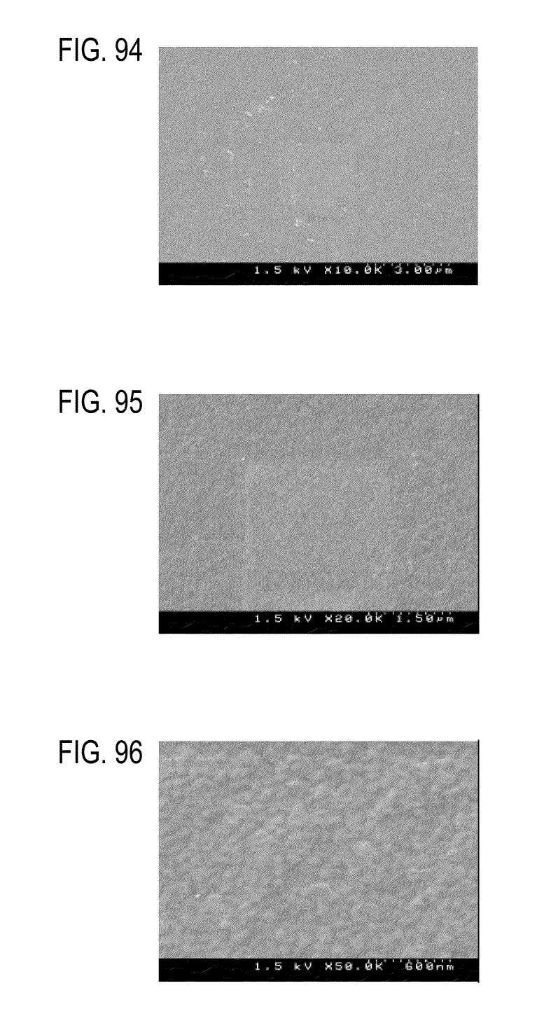

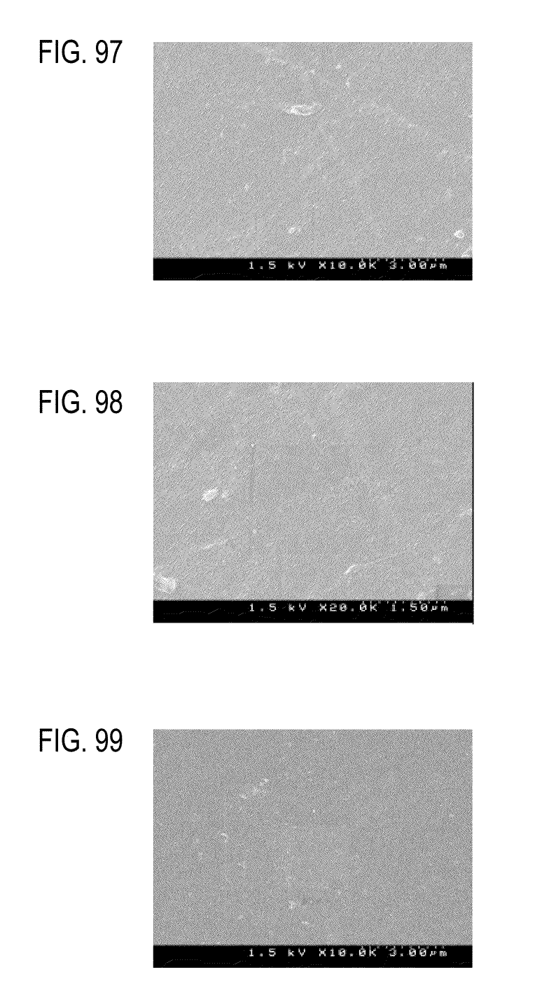

By mixing DNA of 10 mmol/l concentration and PNIPAAm of 50 mmol/l concentration, a film was produced. FIG. 94 is a surface image of a film composed of a mixture of DNA and PNIPAAm observed with SEM at 10,000-fold magnification, FIG. 95 is a surface image of a film composed of a mixture observed at 20,000-fold magnification, and FIG. 96 is a surface image of a film composed of a mixture observed at 50,000-fold magnification. Moreover, FIG. 97 is a back side image of a film composed of a mixture of DNA and PNIPAAm observed with SEM at 10,000-fold magnification, FIG. 98 is a back side image of a film composed of a mixture observed at 20,000-fold magnification. In any magnification, fine pores were not observed on both a surface and a back side.

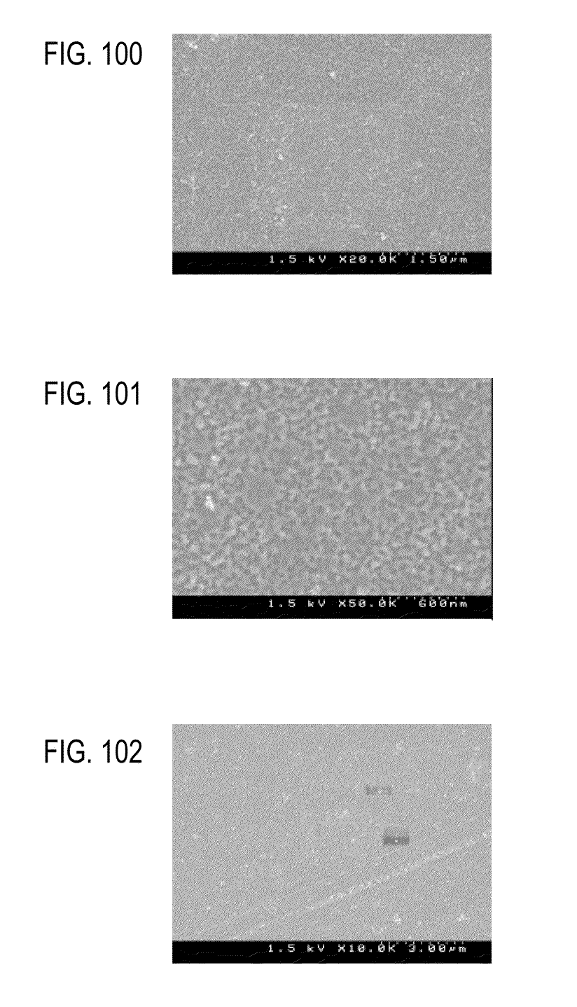

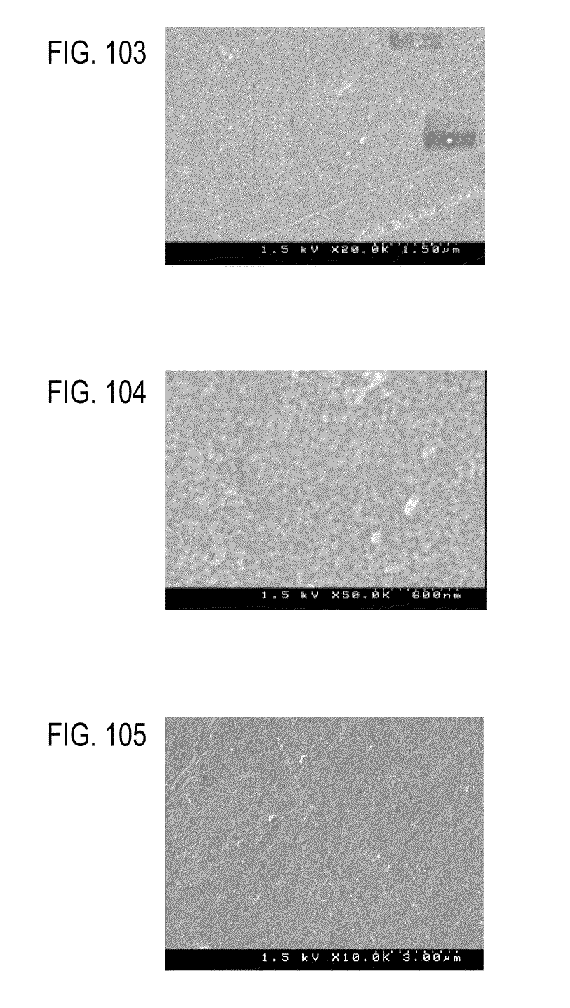

Next, a film composed of a mixture of DNA and PNIPAAm was set in ethanol, PNIPAAm was dissolved, and a porous material according to a sixth example of a fifth embodiment was produced. FIG. 99 is a surface image of a porous material observed at 10,000-fold magnification, FIG. 100 is a surface image of a porous material observed at 20,000-fold magnification, and FIG. 101 is a surface image of a porous material observed at 50,000-fold magnification. A surface became rough with an ethanol treatment, and fine pores were observed. FIG. 102 is a back side image of a porous material observed at 10,000-fold magnification, FIG. 103 is a back side image of a porous material observed at 20,000-fold magnification, and FIG. 104 is a back side image of a porous material observed at 50,000-fold magnification. On a back side, fine pores were also observed.

### Seventh Example of Fifth Embodiment

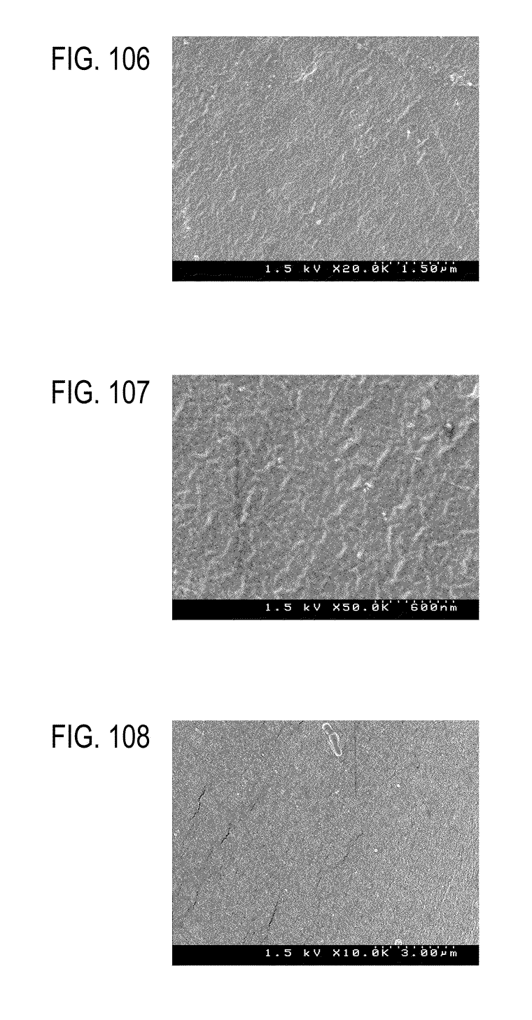

By mixing DNA and polyvinylpyrrolidone (PVP) in a ratio of 2 to 1 concentration, a film was produced. FIG. 105 is a surface image of a film composed of a mixture of DNA and PVP observed with SEM at 10,000-fold magnification. FIG. 106 is a surface image of a film composed of a mixture observed at 20,000-fold magnification, and FIG. 107 is a surface image of a film composed of a mixture observed at 50,000-fold magnification. In any magnification, fine pores were not observed.

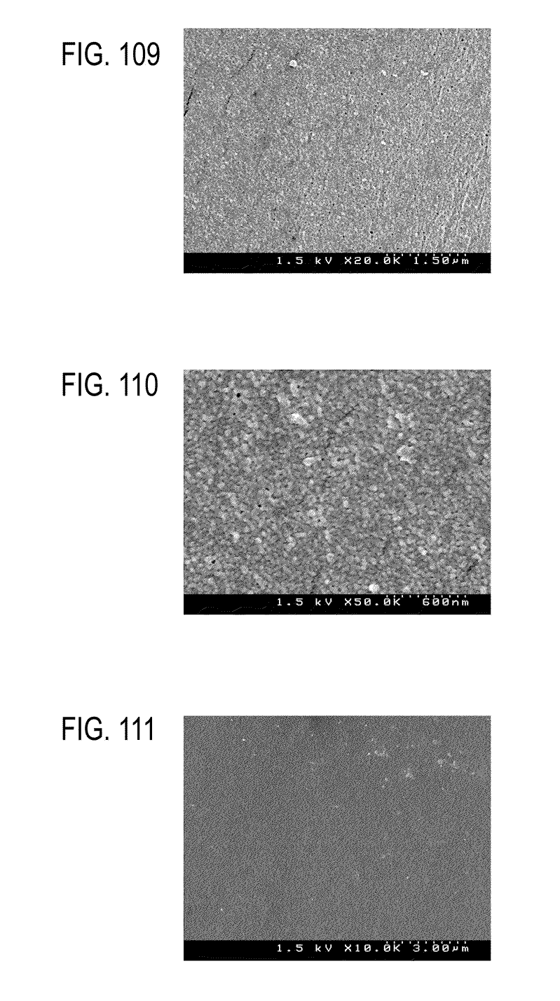

Next, a film composed of a mixture of DNA and PVP was set in ethanol, PVP was dissolved, and a porous material according to a seventh example of a fifth embodiment was produced. FIG. 108 is a surface image of a porous material observed at 10,000-fold magnification, FIG. 109 is a surface image of a porous material observed at 20,000-fold magnification, and FIG. 110 is a surface image of a porous material observed at 50,000-fold magnification. A surface became rough with an ethanol treatment, and fine pores were observed in FIG. 109 and FIG. 110.

### Eighth Example of Fifth Embodiment

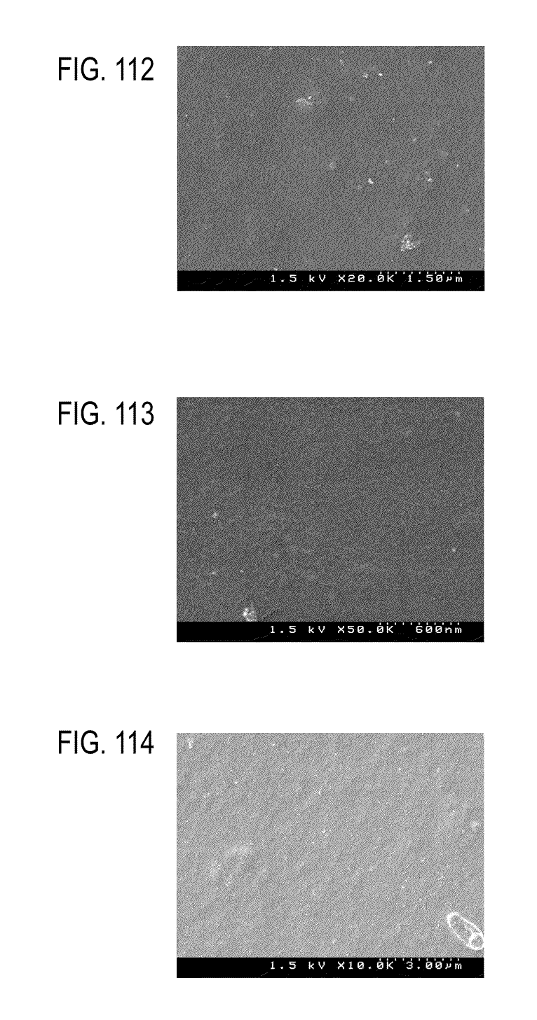

By mixing DNA and PVP in a ratio of 1 to 1 concentration, a film was produced. FIG. 111 is a surface image of a film composed of a mixture of DNA and PVP observed with SEM at 10,000-fold magnification, FIG. 112 is a surface image of a film composed of a mixture observed at 20,000-fold magnification, and FIG. 113 is a surface image of a film composed of a mixture observed at 50,000-fold magnification. In any magnification, fine pores were not observed.

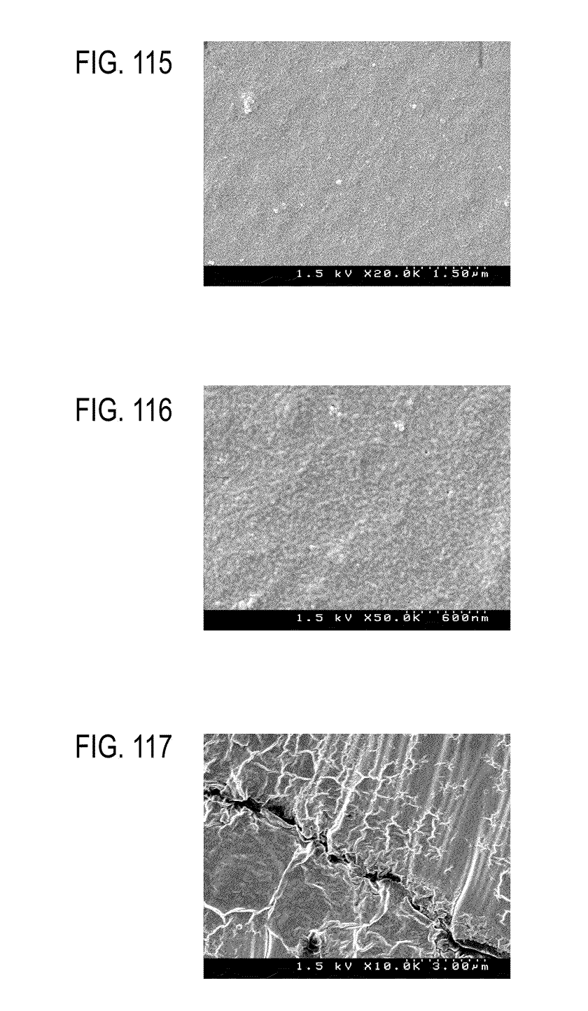

Next, a film composed of a mixture of DNA and PVP was set in ethanol, PVP was dissolved, and a porous material according to an eighth example of a fifth embodiment was produced. FIG. 114 is a surface image of a porous material observed at 10,000-fold magnification, FIG. 115 is a surface image of a porous material observed at 20,000-fold magnification, and FIG. 116 is a surface image of a porous material observed at 50,000-fold magnification. A surface was observed to become rough by an ethanol treatment.

### Ninth Example of Fifth Embodiment

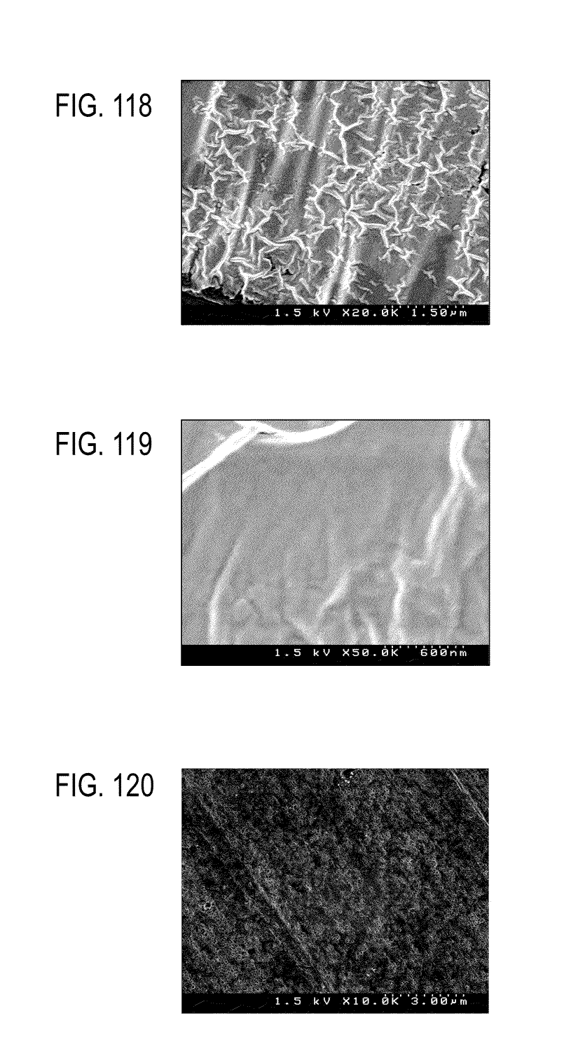

By mixing DNA and PVP in a ratio of 1 to 2 concentration, a film was produced. FIG. 117 is a surface image of a film composed of a mixture of DNA and PVP observed with SEM at 10,000-fold magnification, FIG. 118 is a surface image of a film composed of a mixture observed at 20,000-fold magnification, and FIG. 119 is a surface image of a film composed of a mixture observed at 50,000-fold magnification. In any magnification, fine pores were not observed.

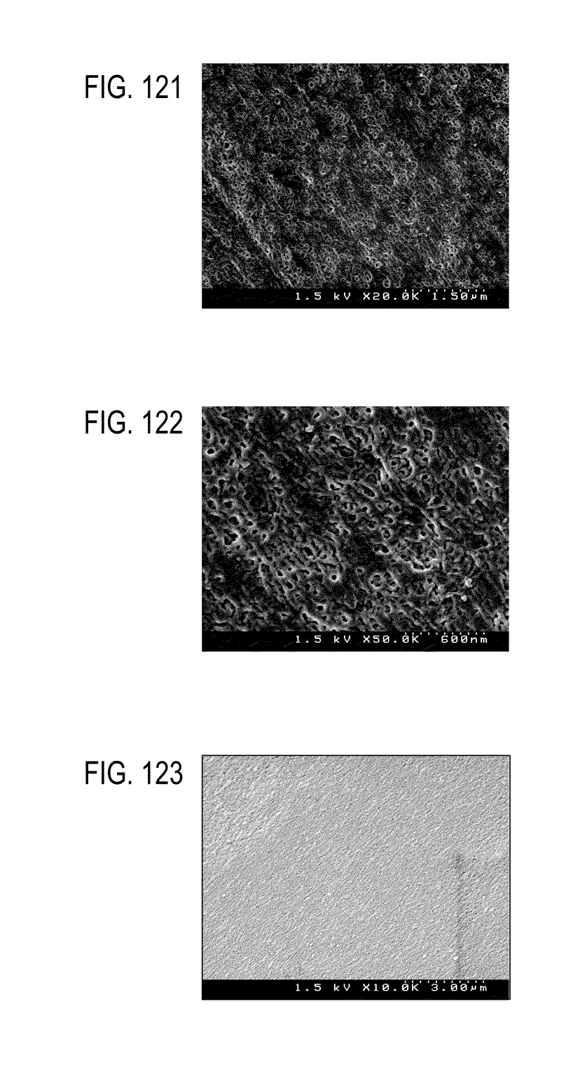

Next, a film composed of a mixture of DNA and PVP was set in ethanol, PVP was dissolved, and a porous material according to a ninth example of a fifth embodiment was produced. FIG. 120 is a surface image of a porous material observed at 10,000-fold magnification, FIG. 121 is a surface image of a porous material observed at 20,000-fold magnification, and FIG. 122 is a surface image of a porous material observed at 50,000-fold magnification. In any image, fine pores were observed.

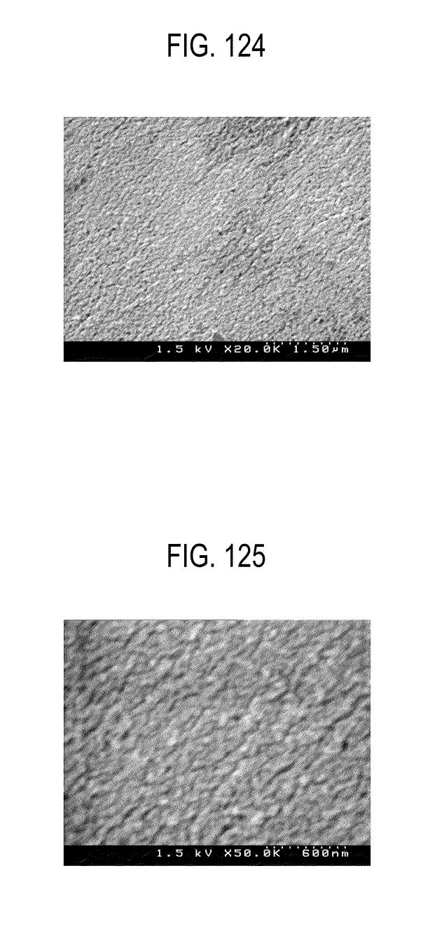

FIG. 123 is a back side image of a porous material observed at 10,000-fold magnification, FIG. 124 is a back side image of a porous material observed at 20,000-fold magnification, and FIG. 125 is a back side image of a porous material observed at 50,000-fold magnification. Fine pores were also observed on a back side, though the number of fine pores was less than that on a surface.

### Sixth Embodiment

A porous material according to a sixth embodiment is composed of DNA ionically-cross-linked with calcium ions (Ca²⁺) or magnesium ions (Mg²⁺), and fine pores of 1 nm to 100 nm in diameter is prepared. A porous material composed of DNA ionically-cross-linked with calcium ions and a porous material composed of DNA ionically-cross-linked with magnesium ions have biocompatibility, respectively, high affinity for a cell, and are useful for a cell culture membrane. Moreover, porous materials according to a sixth embodiment are not dissolved in a cell culture medium for two days or more, and their forms can be preserved.

Next, a production method for a porous material according to a sixth embodiment will be explained. First, in a similar way to a production method for a porous material according to a fifth embodiment shown in FIG. 39, a porous material composed of DNA is formed. After that, a calcium chloride (CaCl₂) aqueous solution of 1 mol/l is dropped on a petri dish, and a porous material is immersed in a CaCl₂ aqueous solution for one day at room temperature. By immersing a porous material in a CaCl₂ aqueous solution, sodium ions are substituted with calcium ions, and DNA is ionically-cross-linked with calcium ion. A gel state of ionically-cross-linked DNA is collected as a porous material according to a sixth embodiment. Note that, instead of a CaCl₂ aqueous solution, by immersing a porous material in a magnesium chloride (MgCl₂) aqueous solution of 1 mol/l, a porous material composed of ionically-cross-linked DNA with magnesium ions can be produced. After collecting a porous material, a porous material may be rinsed with ultrapure water, and dried.

### Example of Sixth Embodiment

First, a fetal bovine serum (FBS) is added to a dulbecco modified eagle medium (DMEM) so as to be 10% in volume concentration, further, adding to penicillin streptomycin so as to be 1%, and a cell culture medium is prepared. Next, a cell cryopreserved solution 62 containing mouse chondrocytes (ATDC5) frozen at −80° C. in a cryopreservation tube 61 as shown in FIG. 2 was defrosted. A defrosted cell cryopreserved medium 62 was dropped into a bottle 63 shown in FIG. 3, further, a cell culture medium was dropped into the bottle 63. After that, mouse chondrocytes were suspended with a cell culture medium, and a first suspension 64 was obtained.

The bottle 63 was rotated at a rotation speed of 2,000 rpm for five minutes, as shown in FIG. 4, mouse chondrocytes 65 contained in the first suspension 64 were precipitated. Next, mouse chondrocytes 65 precipitated were suspended with a cell culture medium, and a second suspension was obtained. After that, as shown in FIG. 5, a second suspension 67 was dropped on a petri dish 66 of 100 mm in diameter, mouse chondrocytes were cultured at 37° C. in a carbon dioxide (CO₂) concentration of 5%. After mouse chondrocytes became confluent on a base plane of the petri dish 66, a cell culture medium in the petri dish 66 was removed with suction. Next, mouse chondrocytes were washed with a phosphate buffered saline (PBS), and after washing, a phosphate buffered saline was removed with suction. After that, trypsin-EDTA of 5 ml was dropped on the petri dish 66, and incubated at 37° C. for 5 minutes. Mouse chondrocytes exfoliated by trypsin-EDTA from the petri dish 66 were centrifuged, and suspended with a cell culture medium.

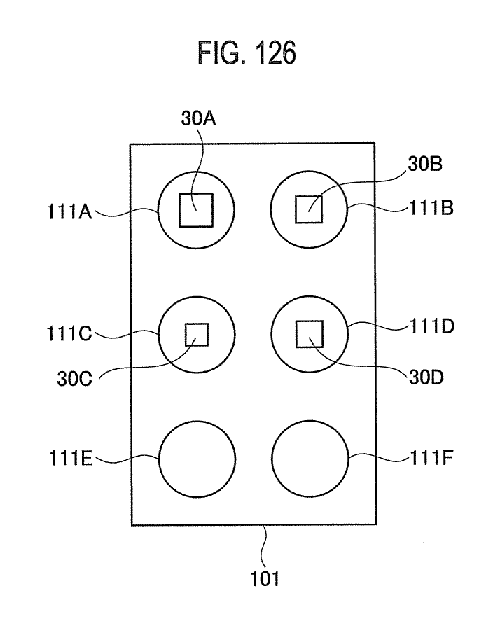

Next, as shown in FIG. 6, a petri dish 101 having six wells 111A, 111B, 111C, 111D, 111E, and 111F was prepared. Moreover, as shown in FIG. 126, porous materials 30A, 30B, 30C and 30D composed of DNA ionically-cross-linked with calcium ions were set on base planes of wells 111A, 111B, 111C, and 111D, respectively. Note that, surface areas of porous materials of 30B and 30C were equal, and surface areas of porous materials of 30B and 30C were smaller than a surface area of porous material 30A. Moreover, a surface area of porous material 30D was smaller than surface areas of porous materials of 30B and 30C. After that, the wells 111A to 111F were filled with a cell culture medium, respectively, and dispersed with mouse chondrocytes.

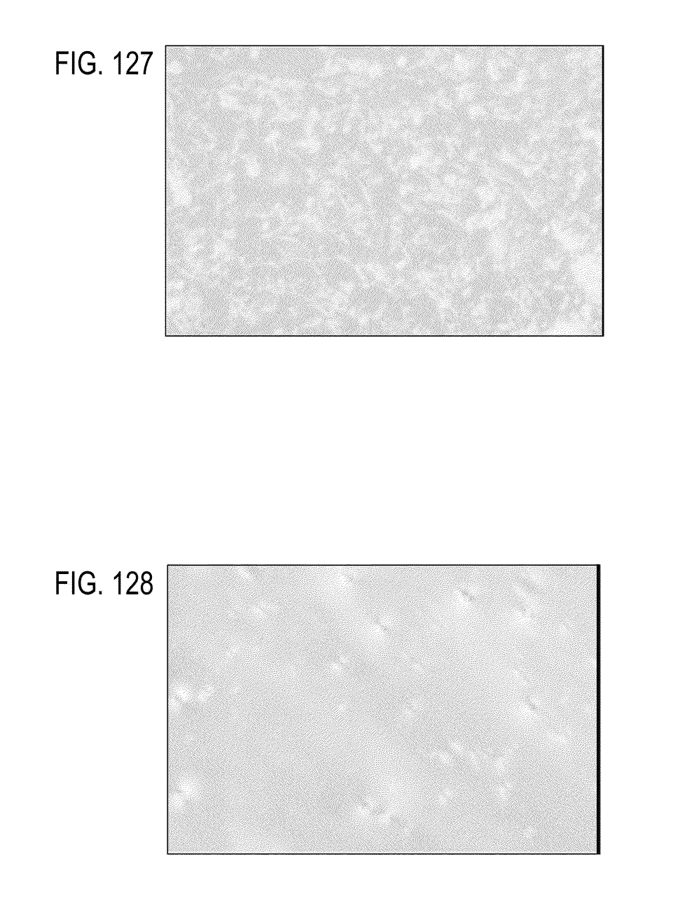

Mouse chondrocytes were adhered to the porous materials of 30A, 30B, 30C, and 30D composed of DNA ionically-cross-linked with calcium ions. Moreover, as shown in FIG. 127, after five days from start of culture, mouse chondrocytes on the porous materials of 30A, 30B, 30C, and 30D increased. Moreover, a difference of adhesion and increase of mouse chondrocytes due to a difference of a surface area of porous materials of 30A, 30B, 30C, and 30D were not observed. As a result, it was shown that a porous material composed of DNA ionically-cross-linked with calcium ions had biocompatibility. Hence, it was shown that a porous material composed of DNA ionically-cross-linked with calcium ions could be transplanted into a living body.

### Comparative Example of Sixth Embodiment

First, a sodium salt of DNA was dissolved in ultrapure water, and a DNA aqueous solution of 7.5 g/l concentration was prepared. Next, a DNA aqueous solution was dropped on a plane such as a basal plane of petri dish of 100 mm in diameter and the like, and by drying a DNA aqueous solution, an water-soluble film composed of a sodium salt of DNA was formed on a basal plane of petri dish. After that, a calcium chloride (CaCl₂) aqueous solution of 1 mol/l was dropped on a petri dish, an water-soluble film was immersed in a CaCl₂ aqueous solution for three hours or more. By immersing an water-soluble film in the CaCl₂ aqueous solution, sodium ions were substituted with calcium ions, and DNA was ionically-cross-linked with calcium ions. A gelled DNA ionically-cross-linked with calcium ions was collected as a flat film according to a comparative example. A flat film according to a comparative example had not fine pores different from a porous material according to an embodiment.

In the same condition as that of an example of a sixth embodiment, mouse chondrocytes were cultured on a flat film according to a comparative example. After five days from start of culture, as shown in FIG. 128, from an observation result of mouse chondrocytes on a flat film, the number of increase of mouse chondrocyte was small compared with that in FIG. 127. Hence, it was shown that a porous material according to an example of a sixth embodiment was excellent in an adhesiveness as well as a cell increase due to a large surface area.

### Seventh Embodiment

A cell culture membrane according to a seventh embodiment is composed of DNA ionically-cross-linked with calcium ions (Ca²⁺) or magnesium ions (Mg²⁺), a porous material having fine pores of 1 nm to 100 nm in diameter, and collagens being arranged in fine pores of a porous material. A cell culture membrane according to a seventh embodiment has higher affinity for a cell, since collagens are arranged in fine pores of a porous material.

Next, a production method for a cell culture membrane according to a seventh embodiment will be explained. First, in the same way as a sixth embodiment, a porous material ionically-cross-linked with calcium ions or magnesium ions is produced. After that, a porous material is immersed in an aqueous solution of salmon-derived type I collagen of 5% weight concentration for one night, and collagens are impregnated into fine pores of a porous material, and a cell culture membrane according to a seventh embodiment is obtained. Note that, DNA may be cross-linked by ultraviolet (UV) irradiation.

### Eighth Embodiment

A porous material according to an eighth embodiment is composed of a DNA photoproduct, and fine pores of 1 nm to 100 nm in diameter are prepared. A DNA photoproduct means a salt of deoxyribonucleic acid and monovalent cations cross-linked by ultraviolet irradiation. A DNA photoproduct is insoluble to water (H₂O). A porous material according to an eighth embodiment is insoluble to a solution and porous at the same time, and is useful as a cell culture membrane, an adsorbent, or a filter for a solution.

Next, a production method for a porous material according to a sixth embodiment will be explained. First, in the same way as a fifth embodiment, a porous material composed of DNA is formed. After that, UV of 1×10⁻² mJ/mm² or more is irradiated to a porous material. With UV irradiation, for example, thymines in DNA contained in a porous material are cross-linked with each other, and thymine dimer is formed. Moreover, with UV irradiation, a part of enzyme of thymine (quinone structure) is cross-linked by reacting with other thymine or cytosine. Or a carbon-hydrogen bond (C—H) of ribose contained in DNA is cut with UV irradiation, a radical carbon reacts with other carbon-hydrogen bonds. As a result, a cell culture membrane is formed from a salt of DNA photo-cross-linked with monovalent cations.

### First Example of Eighth Embodiment

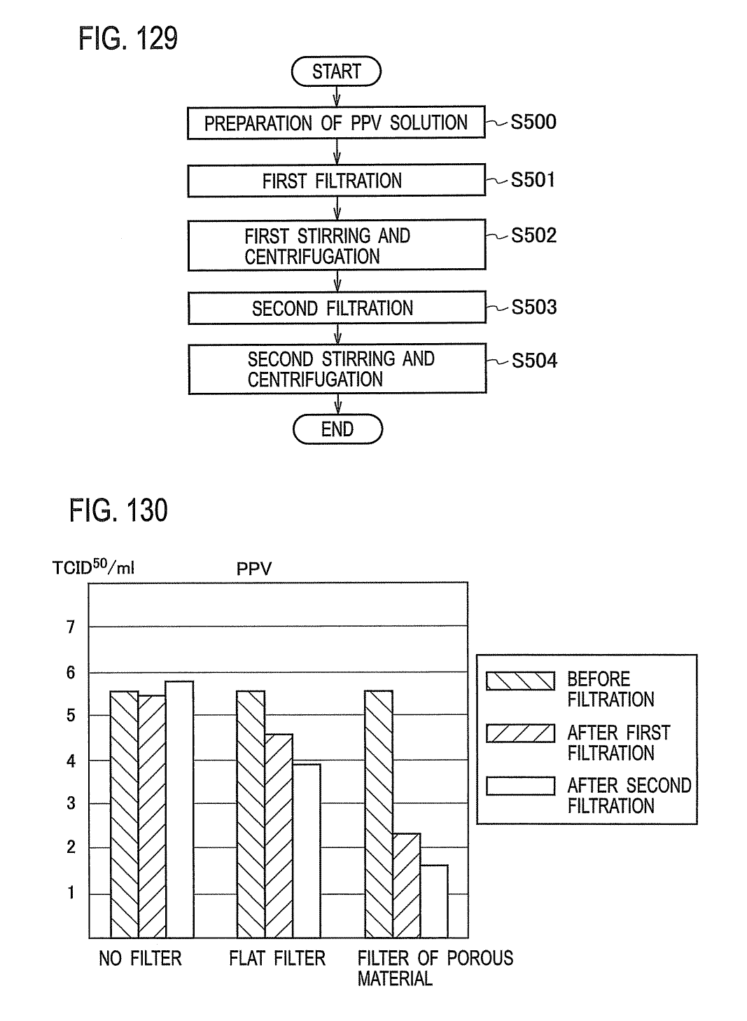

A usefulness of a porous material according to an eighth embodiment as a filter was checked by using porcine parvovirus (PPV). In the following, an explanation will be made in reference to a flow chart shown in FIG. 129.

(a) At a step S500, porcine parvovirus was provided. Porcine parvovirus exists in a pig as a natural host, and is classified into Parvoviridae, Parvovirus. PPV is a DNA virus of a linear single strand not having lipid membranes, and has an extremely high physical and chemical resistance. Porcine parvovirus has a regular icosahedral structure of 18 to 24 nm in size. Next, porcine parvovirus of 2 ml was diluted with ultrapure water of 20 ml so that an viral infectivity became 5.54 TCID⁵⁰/ml, at a step S501, using a porous material according to an eighth embodiment, a solution of 5 ml containing porcine parvovirus was filtrated.

(b) At a step S02, after a filtrate was stirred for 30 minutes, an viral infectivity measured by collecting a supernatant of 1 ml obtained by centrifugation was 2.31 TCID⁵⁰/ml. Hence, an viral infectivity was reduced by 3.23 TCID⁵⁰/ml. Next, at a step S503, a supernatant of 4 ml was filtrated again with a porous material according to an eighth embodiment. After that, a filtrate was stirred for 30 minutes, an viral infectivity measured by collecting a supernatant obtained by centrifugation was 1.61 TCID⁵⁰/ml. Hence, an viral infectivity was reduced by 3.93 TCID⁵⁰/ml.

Note that, as a comparative object, when a filtration at a step S501 was not been done, an viral infectivity of a supernatant obtained after stirring and centrifugation was 5.46 TCID⁵⁰/ml. Moreover, when a filtration at a step S503 was also omitted, an viral infectivity of a supernatant obtained after stirring and centrifugation was 5.81 TCID⁵⁰/ml.

Moreover, as a comparative object, a flat film without fine pores composed of DNA UV-cross-linked was provided. By dropping a DNA aqueous solution on a plane, e.g. a basal plane of petri dish and the like, and drying a DNA aqueous solution, an water-soluble film composed of a sodium salt of DNA was formed, then, a flat film was obtained by irradiating an water-soluble film with UV. At a step S01, when a filtration was done using a flat film instead of a porous material, an viral infectivity of a supernatant after processing was 4.58 TCID⁵⁰/ml. Furthermore, at a step S503, when a filtration was done using a flat film instead of a porous material, an viral infectivity of a supernatant obtained by after processing was 3.88 TCID⁵⁰/ml.

As a result, as shown in FIG. 130, it was shown that a porous material according to an eighth embodiment was useful as a filter for porcine parvovirus. Moreover, it was also shown that, compared with a flat film without fine pores, a porous material was excellent in separation of porcine parvovirus from a solution.

### Second Example of Eighth Embodiment

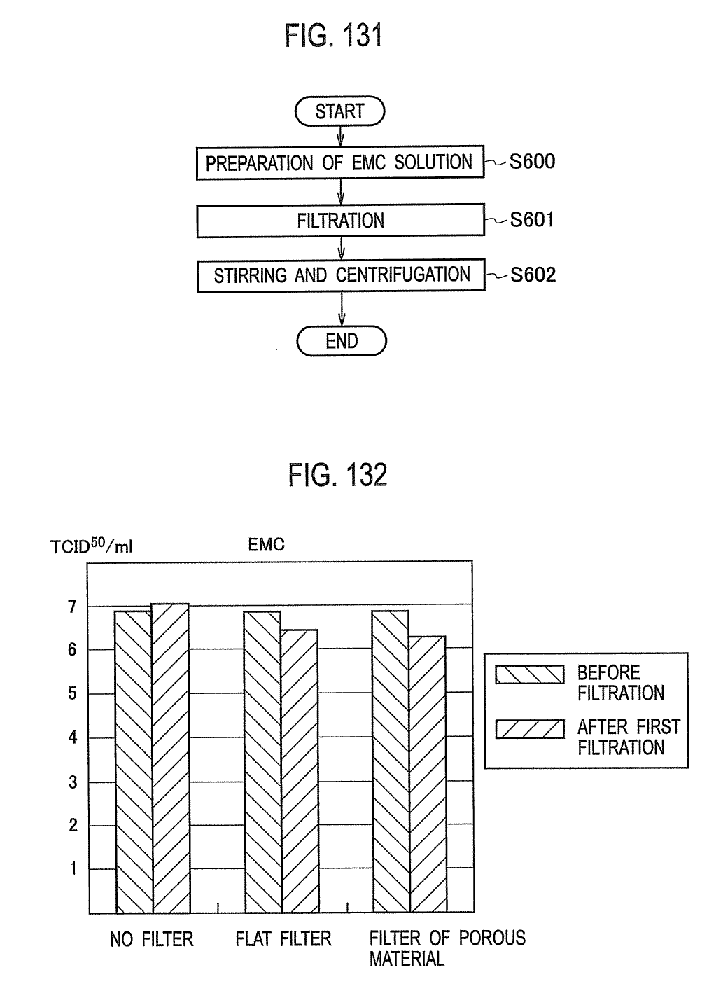

A usefulness as a filter of porous material according to an eighth embodiment was checked using encephalomyocarditis (EMC) virus. In the following, an explanation will be made in reference to a flow chart shown in FIG. 131.

(a) At a step S600, encephalomyocarditis virus was prepared. Encephalomyocarditis virus exists in a mouse as a natural host, is an RNA virus not having lipid membranes classified into Family Picornaviridae, Cardiovirus, and has a moderate physical and chemical resistance. Encephalomyocarditis virus has a regular icosahedral structure of 25 to 30 nm in size. Next, encephalomyocarditis virus of 2 ml was diluted with ultrapure water so that an viral infectivity became 6.85 TCID⁵⁰/ml.

(b) At a step 601, using a porous material according to an eighth embodiment, a solution of 5 ml containing encephalomyocarditis virus was filtrated. At a step 602, after a filtrate was stirred for 30 minutes, an viral infectivity measured by collecting a supernatant of 1 ml obtained by centrifugation was 6.24 TCID⁵⁰/ml. Hence, an viral infectivity was reduced by 0.61 TCID⁵⁰/ml.

Note that, as a comparative object, when a filtration at a step S601 was not done, an viral infectivity of a supernatant obtained after stirring and centrifugation was 7.03 TCID⁵⁰/ml. Moreover, as a comparative object, at a step 601, when a filtration was done using a flat film instead of a porous material, an viral infectivity of a supernatant obtained by after processing was 6.42 TCID⁵⁰/ml.

As a result, as shown in FIG. 132, it was shown that a porous material according to an eighth embodiment was useful as a filter for encephalomyocarditis virus.

### Ninth Embodiment

A porous material according to a ninth embodiment is composed of DNA ionically-cross-linked with quaternary ammonium ions having long-chain alkyl groups as shown in chemical formula (1), and fine pores of 1 nm to 100 μm in diameter were prepared. Note that, when a carbon number in an alkyl group is 13 or more, a quaternary ammonium ion shown in chemical formula (1) has cytotoxicity. Hence, when a porous material according to a ninth embodiment is used as a cell culture membrane, a carbon number in an alkyl group is suitable to make 6 to 12. Moreover, collagens may be arranged in fine pores.

Next, a production method for a porous material according to a ninth embodiment will be explained. First, in the same way as a production method for a porous material according to a fifth embodiment as shown in FIG. 39, a porous material composed of DNA is formed. After that, an aqueous solution containing quaternary ammonium ions is dropped on a petri dish, a porous material is immersed in an aqueous solution containing quaternary ammonium ions for one day at room temperature. By immersing a porous material in an aqueous solution containing quaternary ammonium ions, sodium ions are substituted with quaternary ammonium ions, and DNA is ionically-cross-linked with quaternary ammonium ions. A gelled DNA ionically cross-linked with quaternary ammonium ions is collected as a porous material according to a ninth embodiment.

### Example of Ninth Embodiment

A cytotoxicity test of a porous material composed of DNA ionically-cross-linked with quaternary ammonium ions was done. A cytotoxicity test was followed a separate attachment [Fundamental idea of biological safety evaluation for medical instruments] in [Fundamental idea of biological safety test necessary for approval application of manufacturing medical devices (in port)] (Pharmaceutical and Medical Safety Bureau Ministry of Hearth notification No. 0213001, 2003). Concretely, a porous material shaped as a semicircular film was set so as to occupy on a half space of circular basal plane of a petri dish. Next, a check was done whether V79 cells increased on both of basal planes of petri dish to form a colony or not. As a result, V79 cells increased on a porous material composed of DNA ionically-cross-linked with quaternary ammonium ions having a carbon number of 6 to 12 in an alkyl group, and colonies were formed. However, V79 cells did not increased on a porous material composed of DNA ionically-cross-linked with quaternary ammonium ions having a carbon number of 13 or more in an alkyl group.

### Other Embodiment

As described above, the present invention was described by embodiments, the description and the figures constituting a part of this disclosure should not be understood as those to limit this invention. From this disclosure, for the person skilled in the art, various alternative embodiments, examples, and an application technology should become clear. For example, in a first and a fifth embodiments, a method for purifying DNA by using enzymes was shown. In contrast, sodium chloride (NaCl) and sodium dodecyl sulfate (SDS) may be added to a first filtrate obtained in a step S100 in FIG. 1 and a step S300 in FIG. 39. After that, a first filtrate is heated at 60° C. to 80° C., a supernatant is obtained by centrifugation of a first filtrate. By adding ethanol to a supernatant, a sodium salt of DNA is precipitated. In this way, the present invention should be understood to include various embodiments not described here. Hence, the present invention should only be limited to an identity of invention of claims being acceptable from this disclosure.

### INDUSTRIAL APPLICABILITY

The present invention has usefulness of providing a fundamental medical treatment of a disease difficult to be treated up to now (regenerative medicine), technology, and medicine. Moreover, the present invention has possibilities of application to a filter and the like for a virus and so on.

1. A cell culture membrane comprising deoxyribonucleic acid ionically-cross-linked with calcium ions.

2. A cell culture membrane comprising deoxyribonucleic acid ionically-cross-linked with magnesium ions.

3. The cell culture membrane, according to claim 1, further comprising collagen.

4. A cell culture membrane comprising a salt of deoxyribonucleic acid and cations cross-linked by ultraviolet irradiation.

5. A cell culture kit comprising a cell culture membrane comprising deoxyribonucleic acid ionically-cross-linked with calcium ions, wherein cells are adhered to the cell culture membrane.

6. A cell culture kit comprising a cell culture membrane comprising deoxyribonucleic acid ionically-cross-linked with magnesium ions, wherein cells are adhered to the cell culture membrane.

7. A cell culture kit comprising a cell culture membrane comprising a salt of deoxyribonucleic acid and cations cross-linked by ultraviolet irradiation, wherein cells are adhered to the cell culture membrane.

8. A production method for a cell culture membrane, comprising:
forming an water-soluble film containing a salt of deoxyribonucleic acid and monovalent cations; and
ionically-cross-linking the deoxyribonucleic acid with the calcium ions by substituting the monovalent cations with calcium ions.

9. The production method for the cell culture membrane, according to claim 8, wherein substituting the monovalent cations with the calcium ions contains immersing the water-soluble film in an aqueous solution containing the calcium ions.

10. A production method for a cell culture membrane comprising:
forming an water-soluble film containing a salt of deoxyribonucleic acid and monovalent cations; and
ionically-cross-linking the deoxyribonucleic acid with the magnesium ions by substituting the monovalent cations with magnesium ions.

11. The production method for the cell culture membrane, according to claim 10, wherein substituting the monovalent cations with the magnesium ions contains immersing the water-soluble film in an aqueous solution containing the magnesium ions.

12. The production method for the cell culture membrane, according to claim 8, wherein the monovalent cations are sodium ions.

13. The production method for the cell culture membrane, according to claim 8, further comprising:
immersing the water-soluble film in an aqueous solution of collagen.

14. The production method for the cell culture membrane, according to claim 8, further comprising:
immersing deoxyribonucleic acid ionically-cross-linked with the calcium ions in an aqueous solution of collagen.

15. The production method for the cell culture membrane, according to claim 10, further comprising:
immersing deoxyribonucleic acid ionically-cross-linked with the magnesium ions in an aqueous solution of collagen.

16. A production method for a cell culture membrane comprising:
forming an water-soluble film containing a salt of deoxyribonucleic acid and monovalent cations; and
irradiating the water-soluble film with ultraviolet.

17. A cell culture membrane comprising deoxyribonucleic acid ionically-cross-linked with quaternary ammonium ions.

18. The cell culture membrane, according to claim 17, wherein a carbon number in an alkyl group of the quaternary ammonium ions is 6 to 12.

19. A cell culture kit comprising a cell culture membrane comprising deoxyribonucleic acid ionically-cross-linked with quaternary ammonium ions, wherein cells are adhered to the cell culture membrane.

20. A production method for a cell culture membrane comprising:
forming an water-soluble film containing a salt of deoxyribonucleic acid and sodium ions; and
ionically-cross-linking the deoxyribonucleic acid with the quaternary ammonium ions by substituting the sodium ions with quaternary ammonium ions.

21. The production method for the cell culture membrane, according to claim 20, wherein a carbon number in an alkyl group of the quaternary ammonium ions is 6 to 12.

22. A porous material having fine pores of 1 nm to 100 μm in diameter, comprising deoxyribonucleic acid.

23. The porous material, according to claim 22, wherein the deoxyribonucleic acid is cross-linked.

24. A cell culture membrane comprising:
a porous material having fine pores of 1 nm to 100 μm in diameter, comprising cross-linked deoxyribonucleic acid; and
collagen arranged in the fine pores of the porous material.

25. A production method for a porous material, comprising:
preparing a mixture by mixing deoxyribonucleic acid and an alcohol soluble polymer; and
forming a porous material composed of the deoxyribonucleic acid by dissolving the alcohol soluble polymer contained in the mixture with alcohol.

26. The production method for the porous material, according to claim 25, wherein the alcohol is ethanol.

27. The production method for the porous material, according to claim 25, wherein the alcohol soluble polymer is poly(N-isopropyl acrylamide).

28. The production method for the porous material, according to claim 25, further comprising:
ionically-cross-linking the deoxyribonucleic acid contained in the porous material with calcium ions.

29. The production method for the porous material, according to claim 25, further comprising:
ionically-cross-linking the deoxyribonucleic acid contained in the porous material with magnesium ions.

30. The production method for the porous material, according to claim 25, further comprising:
cross-linking the deoxyribonucleic acid by irradiating the deoxyribonucleic acid contained in the porous material with ultraviolet.

31. The production method for the porous material, according to claim 25, further comprising:
ionically-cross-linking the deoxyribonucleic acid contained in the porous material with quaternary ammonium ions.

32. The production method for the porous material, according to claim 31, wherein a carbon number in an alkyl group of the quaternary ammonium ions is 6 to 12.

33. A production method for a cell culture membrane comprising:
preparing a mixture by mixing deoxyribonucleic acid and an alcohol soluble polymer;
forming a porous material composed of the deoxyribonucleic acid by dissolving the alcohol soluble polymer contained in the mixture with alcohol;
cross-linking the deoxyribonucleic acid contained in the porous material; and
impregnating collagen into the porous material.

34. The production method for the cell culture membrane, according to claim 33, wherein the alcohol is ethanol.

35. The production method for the cell culture membrane, according to claim 33, wherein the alcohol soluble polymer is poly(N-isopropyl acrylamide).
